(19) United States
(12) Patent Application Publication
(10) Pub. No.: US 2004/0091929 A1
(43) Pub. Date: May 13, 2004

(54) Methods and compositions for the diagnosis and treatment of cardiovascular and tumorigenic disease using 4941

(75) Inventors: Katherine A. Galvin, Jamaica Plain, MA (US); Laura A. Rudolph-Owen, Jamaica Plain, MA (US)

Correspondence Address: MILLENNIUM PHARMACEUTICALS, INC., 75 Sidney Street, Cambridge, MA, 02139, US

(73) Assignee: Millennium Pharmaceuticals, Inc.

(21) Appl. No.: 10/696,706

(22) Filed: Oct. 29, 2003

Related U.S. Application Data
(63) Continuation of application No. 09/635,521, filed on Aug. 9, 2000, now abandoned.
(60) Provisional application No. 60/199,908, filed on Apr. 26, 2000.

(51) Int. Cl. C12Q 1/68

(52) U.S. Cl. 435/6

(57) ABSTRACT

The present invention relates to methods and compositions for the diagnosis and treatment of cardiovascular disease, including, but not limited to, atherosclerosis, reperfusion injury, hypertension, restenosis, arterial inflammation, and endothelial cell disorders, such as disorders associated with aberrant endothelial cell growth, angiogenesis and/or vascularization, e.g., tumorigenic disorders. Specifically, the present invention identifies GPCR 4941 genes which are differentially expressed in cardiovascular disease states, relative to their expression in normal, or non-cardiovascular disease states, and/or in response to manipulations relevant to cardiovascular disease. The present invention also identifies GPCR 4941 genes as differentially expressed in tumorigenic disease, e.g., ovarian cancer. The present invention describes methods for the diagnostic evaluation and prognosis of various cardiovascular and tumorigenic diseases, and for the identification of subjects exhibiting a predisposition to such conditions. The present invention provides methods for the diagnostic monitoring of patients undergoing clinical evaluation for the treatment of cardiovascular disease and tumorigenic, and for monitoring the efficacy of compounds in clinical trials. The present invention also provides methods for the identification and therapeutic use of compounds as treatments of cardiovascular and tumorigenic disease.

### RELATED APPLICATIONS

This application claims priority to U.S. Provisional Patent Application No. 60/199,908 filed on Apr. 26, 2000, incorporated herein in its entirety by reference.

### BACKGROUND OF THE INVENTION

Cardiovascular disease is a major health risk throughout the industrialized world. Atherosclerosis, the most prevalent of cardiovascular diseases, is the principal cause of heart attack, stroke, and gangrene of the extremities, and thereby the principle cause of death in the United States. Atherosclerosis is a complex disease involving many cell types and molecular factors (described in, for example, Ross, 1993, Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibro'proliferative response to numerous different forms of insult. Injury or dysfunction of the vascular endothelium is a common feature of may conditions that predispose an individual to accelerated development of atherosclerotic cardiovascular disease. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque.

Such plaques occlude the blood vessel concerned and, thus, restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults.

Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis.

The principal surgical approaches to the treatment of ischemic atherosclerosis are bypass grafting, endarterectomy, and percutaneous translumenal angioplasty (PCTA). The failure rate after these approaches due to restenosis, in which the occlusions recur and often become even worse, is extraordinarily high (30-50%). It appears that much of the restenosis is due to further inflammation, smooth muscle accumulation, and thrombosis. Additional therapeutic approaches to cardiovascular disease have included treatments that encouraged angiogenesis in such conditions as ischemic heart and limb disease.

Angiogenesis is a fundamental process by which new blood vessels are formed, as reviewed, for example, by Folkman and Shing, J. Biol. Chem. 267:10931-10934 (1992). Capillary blood vessels consist of endothelial cells and pericytes. These two cell types carry all of the genetic information to form tubes, branches and whole capillary networks. Specific angiogenic molecules and growth factors can initiate this process. Specific inhibitory molecules can stop it. These molecules with opposing function appear to be continuously acting in concert to maintain a stable microvasculature in which endothelial cell turnover is thousands of days. However, the same endothelial cells can undergo rapid proliferation, i.e. less than five days, during burst of angiogenesis, for example, during wound healing.

Key components of the angiogenic process are the degradation of the basement membrane, the migration and proliferation of capillary endothelial cell (EC) and the formation of three dimensional capillary tubes. The normal vascular turnover is rather low: the doubling time for capillary endothelium is from 50-20,000 days, but it is 2-13 days for tumor capillary endothelium. The current understanding of the sequence of events leading to angiogenesis is that a cytokine capable of stimulating endothelial cell proliferation, such as fibroblast growth factor (FGF), causes release of collagenase or plasminogen activator which, in turn, degrade the basement membrane of the parent venule to facilitate the migration of the endothelial cells. These capillary cells, having sprouted from the parent vessel, proliferate in response to growth factors and angiogenic agents in the surrounding environment to form lumen and eventually new blood vessels.

The development of a vascular blood supply is essential in reproduction, development and wound repair (Folkman, et al., Science 43:1490-1493 (1989)). Under these conditions, angiogenesis is highly regulated, so that it is turned on only as necessary, usually for brief periods of days, then completely inhibited. However, a number of serious diseases' are also dominated by persistent unregulated angiogenesis and/or abnormal neovascularization including solid tumor growth and metastasis, psoriasis, endometriosis, Grave's disease, ischemic disease (e.g., atherosclerosis), and chronic inflammatory diseases (e.g., rheumatoid arthritis), and some types of eye disorders, (reviewed by Auerbach, et al., J. Microvasc. Res. 29:401-411 (1985); Folkman, Advances in Cancer Research, eds. Klein and Weinhouse, pp. 175-203 (Academic Press, New York, 1985); Patz, Am. J. Opthalmol. 94:715-743 (1982); and Folkman, et al., Science 221:719-725 (1983)). For example, there are a number of eye diseases, many of which lead to blindness, in which ocular neovascularization occurs in response to the diseased state. These ocular disorders include diabetic retinopathy, macular degeneration, neovascular glaucoma, inflammatory diseases and ocular tumors (e.g., retinoblastoma). There are a number of other eye diseases which are also associated with neovascularization, including retrolental fibroplasia, uveitis, eye diseases associated with choroidal neovascularization and eye diseases which are associated with iris neovascularization.

Ovarian cancer is the second most common cancer of the female reproductive organs and the fourth leading cause of cancer deaths among American women. There are three main types of ovarian tumors: epithelial tumors, germ cell tumors, and stromal cell tumors, based on the kind of cells from which the tumor originates. The majority of ovarian cancers are thought to arise from the ovarian surface epithelium. The ovarian surface epithelium is a highly dynamic tissue which undergoes morphogenic changes. It has significant proliferative properties, as it must proliferate rapidly to cover the ovulatory site after ovulation of the ova. In addition, morphological and histochemical studies suggest that the ovarian surface epithelium has secretory, endocytotic and transport functions which are hormonally controlled (Blaustein and Lee (1979) Oncol. 8:34-43; Nicosia and Johnson (1983) Int. J. Gynecol. Pathol. 3:249-260; Papadaki and Beilby (1971) J. Cell Sci. 8:445-464; Anderson et al. (1976) J. Morphol. 150:135-164). Epithelial ovarian cancer has a distinctive pattern of spread: cancer cells may migrate through the peritoneum to produce multiple metastatic nodules in the visceral and parietal peritoneum and the hemidiaphragms. In addition cancer cells metastasize through the lymphatic and blood vessels to areas such as the liver, lung and brain.

Since ovarian cancers are generally not readily detectable by diagnostic techniques until the disease has progressed to a late stage of development (Siemens and Auersperg (1988) J. Cellular Physiol. 134:347-356), it is one of the most lethal of the gynecological malignancies. Although a number of potential tumor markers including the cancer antigen 125 (Ca-125) have been evaluated, nonspecificity of the antigens diminish their value as markers for primary ovarian cancer (Kudlacek et al. (1989) Gyn. Onc. 35:323-329; Rustin et al. (1989) J. Clin. Onc. 7:1667-1671; Sevelda et al. (1989) Am. J. Obstet. Gynecol. 161:1213-1216; Omar et al. (1989) Tumor Biol. 10:316-323). Thus, there is a vital need for tumor markers which can be used in the specific, early detection of ovarian cancer, the monitoring of cancer therapies, the immunodetection of ovarian tumors, and the development of probes for potential use in immunotherapy (Cantarow et al. (1981) Int. J. Radiation Oncol. Biol. Phys. 7:1095-1098).

### SUMMARY OF THE INVENTION

The present invention provides methods and compositions for the diagnosis and treatment of cardiovascular disease, including but not limited to, atherosclerosis, ischemia/reperfusion injury, hypertension, restenosis, arterial inflammation, and endothelial cell disorders, such as disorders associated with aberrant endothelial cell growth, angiogenesis and/or vascularization. The present invention also provides methods and compositions for the diagnosis and treatment of tumorigenic disease, e.g., ovarian tumors, and diseases such as diabetic retinopathy, and psoriasis, all being examples of disease states characterized by neo-vascularization.

The present invention is based, at least in part, on the discovery that the GPCR 4941 gene is differentially expressed in endothelial cells treated under conditions of laminar sheer stress (LSS), cytokine stimulation, growth on Matrigel, and proliferation, as well as in an aminal model of atherosclerosis. The present invention is further based, at least in part, on the discover that the GPCR 4941 gene is differentially expressed in tumorigenic disease. In particular, the GPCR 4941 gene is upregulated in ovarian tumors as compared to normal ovary samples.

In one aspect, the invention provides a method for identifying the presence of a nucleic acid molecule associated with a cardiovascular or tumorigenic disorder, in a sample by contacting a sample comprising nucleic acid molecules with a hybridization probe comprising at least 25 contiguous nucleotides of SEQ ID NO:1 or 3, and detecting the presence of a nucleic acid molecule associated with a cardiovascular disorder or tumorigenic disorder, when the sample contains a nucleic acid molecule that hybridizes to the nucleic acid probe. In one embodiment, the hybridization probe is detectably labeled. In another embodiment the sample comprising nucleic acid molecules is subjected to agarose gel electrophoresis and southern blotting prior to contacting with the hybridization probe. In a further embodiment, the sample comprising nucleic acid molecules is subjected to agarose gel electrophoresis and northern blotting prior to contacting with the hybridization probe. In yet another embodiment, the detecting is by in situ hybridization. In other embodiments, the method is used to detect mRNA or genomic DNA in the sample.

The invention also provides a method for identifying a nucleic acid associated with a cardiovascular or tumorigenic disorder, in a sample, by contacting a sample comprising nucleic acid molecules with a first and a second amplification primer, the first primer comprising at least 25 contiguous nucleotides of SEQ ID NO:1 or 3 and the second primer comprising at least 25 contiguous nucleotides from the complement of SEQ ID NO:1 or 3, incubating the sample under conditions that allow for nucleic acid amplification, and detecting the presence of a nucleic acid molecule associated with a cardiovascular or tumorigenic disorder, when the sample contains a nucleic acid molecule that is amplified. In one embodiment, the sample comprising nucleic acid molecules is subjected to agarose gel electrophoresis after the incubation step.

In addition, the invention provides a method for identifying a polypeptide associated with a cardiovascular disorder or tumorigenic disorder, in a sample by contacting a sample comprising polypeptide molecules with a binding substance specific for a GPCR 4941 polypeptide, and detecting the presence of a polypeptide associated with a cardiovascular disorder or tumorigenic disorder, when the sample contains a polypeptide molecule that binds to the binding substance. In one embodiment the binding substance is an antibody. In another embodiment, the binding substance is a GPCR 4941 ligand. In a further embodiment, the binding substance is detectably labeled.

In another aspect, the invention provides a method of identifying a subject at risk for a cardiovascular or tumorigenic disorder, by contacting a sample obtained from the subject comprising nucleic acid molecules with a hybridization probe comprising at least 25 contiguous nucleotides of SEQ ID NO:1 or 3, and detecting the presence of a nucleic acid molecule which identifies a subject a risk for a cardiovascular disorder or tumorigenic disorder, when the sample contains a nucleic acid molecule that hybridizes to the nucleic acid probe.

In a further aspect, the invention provides a method for identifying a subject at risk for a cardiovascular disorder or tumorigenic disorder, by contacting a sample obtained from a subject comprising nucleic acid molecules with a first and a second amplification primer, the first primer comprising at least 25 contiguous nucleotides of SEQ ID NO:1 or 3 and the second primer comprising at least 25 contiguous nucleotides from the complement of SEQ ID NO:1 or 3, incubating the sample under conditions that allow for nucleic acid amplification, and detecting a nucleic acid molecule which identifies a subject at risk for a cardiovascular disorder or tumorigenic disorder, when the sample contains a nucleic acid molecule that is amplified.

In yet another aspect, the invention provides a method of identifying a subject at risk for a cardiovascular disorder or tumorigenic disorder, by contacting a sample obtained from the subject comprising polypeptide molecules with a binding substance specific for a GPCR 4941 polypeptide, and identifying a subject at risk for a cardiovascular disorder or tumorigenic disorder, by detecting the presence of a polypeptide molecule in the sample that binds to the binding substance.

In another aspect, the invention provides a method for identifying a compound capable of treating a cardiovascular or tumorigenic disorder, characterized by aberrant GPCR 4941 nucleic acid expression or GPCR 4941 protein activity by assaying the ability of the compound to modulate the expression of a GPCR 4941 nucleic acid or the activity of a GPCR 4941 protein. In one embodiment, the disorder is an endothelial cell disorder associated with aberrant angiogenesis and/or vascularization. In another embodiment, the disorder is atherosclerosis. In yet another embodiment, the disorder is a tumorigenic disorder, e.g., an ovarian tumor. In a further embodiment, the ability of the compound to modulate the activity of the GPCR 4941 protein is determined by detecting the induction of an intracellular second messenger.

In addition, the invention provides a method for treating a subject having a cardiovascular disorder or tumorigenic disorder, characterized by aberrant GPCR 4941 protein activity or aberrant GPCR 4941 nucleic acid expression by administering to the subject a GPCR 4941 modulator. In one embodiment, the GPCR 4941 modulator is administered in a pharmaceutically acceptable formulation. In another embodiment the GPCR 4941 modulator is administered using a gene therapy vector. In a further embodiment, the GPCR 4941 modulator is a small molecule.

In one embodiment, a GPCR modulator is capable of modulating GPCR 4941 polypeptide activity. In another embodiment, the GPCR 4941 modulator is an anti-GPCR 4941 antibody. In a further embodiment, the GPCR 4941 modulator is a GPCR 4941 polypeptide comprising the amino acid sequence of SEQ ID NO:2, or a fragment thereof. In yet another embodiment, the GPCR 4941 modulator is a GPCR 4941 polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4. In a further embodiment, the GPCR 4941 modulator is an isolated naturally occurring allelic variant of a polypeptide consisting of the amino acid sequence of SEQ ID NO:2, wherein the polypeptide is encoded by a nucleic acid molecule which hybridizes to a complement of a nucleic acid molecule consisting of SEQ ID NO:1 or 3 at 4×SSC at 65-70° C. followed by one or more washes in 1×SSC, at 65-70° C.

In one embodiment, the GPCR 4941 modulator is capable of modulating GPCR 4941 nucleic acid expression. In another embodiment, the GPCR 4941 modulator is an antisense GPCR 4941 nucleic acid molecule. In yet another embodiment, the GPCR 4941 modulator is a ribozyme. In a further embodiment, the GPCR 4941 modulator comprises the nucleotide sequence of SEQ ID NO:1 or 3, or a fragment thereof. In another embodiment, the GPCR 4941 modulator comprises a nucleic acid molecule encoding a polypeptide comprising an amino acid sequence which is at least 90 percent identical to the amino acid sequence of SEQ ID NO:2, wherein the percent identity is calculated using the ALIGN program for comparing amino acid sequences, a PAM120 weight residue table, a gap length penalty of 12, and a gap penalty of 4. In yet another embodiment, the GPCR 4941 modulator comprises a nucleic acid molecule encoding a naturally occurring allelic variant of a polypeptide comprising the amino acid sequence of SEQ ID NO:2, wherein the nucleic acid molecule which hybridizes to a complement of a nucleic acid molecule consisting of SEQ ID NO:1 or 3 at 4×SSC at 65-70° C. followed by one or more washes in 1×SSC, at 65-70° C.

In another aspect, the invention provides a method for identifying a compound capable of modulating an endothelial cell activity by contacting an endothelial cell with a test compound and assaying the ability of the test compound to modulate the expression of a GPCR 4941 nucleic acid or the activity of a GPCR 4941 protein. In certain embodiments, a compound that modulates the expression of a GPCR 4941 nucleic acid or the activity of a GPCR 4941 protein modulates endothelial cell proliferation, migration, or the expression of cell surface adhesion molecules.

Furthermore, the invention provides a method for modulating an endothelial cell activity comprising contacting an endothelial cell with a GPCR 4941 modulator.

Other features and advantages of the invention will be apparent from the following detailed description and claims.

### BRIEF DESCRIPTION OF THE DRAWINGS

FIG. 1 depicts the cDNA sequence and predicted amino acid sequence of human GPCR 4941 (GPR39; GenBank Accession AF034633). The nucleotide sequence corresponds to nucleic acids 1 to 2528 of SEQ ID NO:3. The amino acid sequence corresponds to amino acids 1 to 453 of SEQ ID NO: 2. The coding region without the 5′ and 3′ untranslated region of the human GPCR 4941 gene is shown in SEQ ID NO:1.

FIG. 2 is a graph depicting transcriptional profiling data of GPCR 4941 cDNA expression in human umbilical vein endothelial cells (HUVEC) under static conditions (St), or in response to laminar shear stress (LSS) and Interleukin (IL)-1 stimulation.

FIG. 3 is a graph depicting the results of RT-PCR analysis of GPCR 4941 expression in HUVEC under static conditions (stat), or in response to laminar shear stress (LSS) and Interleukin-1 (IL-1) stimulation.

FIG. 4 is a graph depicting the results of a RT-PCR analysis of GPCR 4941, cyclin B 1, TE-2, FLT, and FLK expression in human microvascular endothelial cells (HMVEC) under control conditions (C), when plated on Matrigel (T), or when plated as confluent layers on tissue culture plastic in the absense of growth factors (A) for 6, 24 or 36 hours.

FIG. 5 is a graph depicting the results of a RT-PCR analysis of GPCR 4941 and cyclin BI expression in HUVEC and HMVEC under conditions of rapid proliferation (P5), confluence/quiescence (C2), or growth factor depletion/quiescence (A24).

FIG. 6 is a graph depicting the results of a RT-PCR analysis of GPCR 4941 expression in the abdominal (Abdm) and arch (Arch) regions of the aorta in ApoE knockout animals at 5, 18, or 33 weeks of age.

FIG. 7 is a graph depicting the results of a quantitative PCR analysis of GPCR 4941 expression in ovarian tumors (T) as compared to normal (N) ovary samples (Panel A); and in endometrioid (endo), mucinous (mucin), and serous (ser) type ovarian tumors as compared to normal ovarian epithelial (norm) samples (Panel B).

FIG. 8 is a graph depicting the results of a quantitative PCR analysis of GPCR 4941 expression in human tumors (T) as compared to normal (N) tissue samples. Panel A, breast, ovary, and lung tumors; Panel B, colon and brain tumors.

### DETAILED DESCRIPTION OF THE INVENTION

The present invention provides methods and compositions for the diagnosis and treatment of cardiovascular disease, including but not limited to, atherosclerosis, ischemia/reperfusion injury, hypertension, restenosis, arterial inflammation, and endothelial cell disorders, such as disorders associated with aberrant endothelial cell growth, angiogenesis and/or vascularization. The present invention also provides methods and compositions for the diagnosis and treatment of tumorigenic disease, e.g., ovarian tumors.

The present invention is based, at least in part, on the discovery that G protein-coupled receptor (GPCR) genes, referred to herein as “G protein-coupled receptor 4941” or “GPCR 4941” nucleic acid and protein molecules, are differentially expressed in cardiovascular disease states relative to their expression in normal, or non-cardiovascular disease states, as well as in endothelial cells treated under conditions of laminar sheer stress (LSS), cytokine stimulation, growth on Matrigel, and proliferation. GPCR 4941 nucleic acid and protein molecules are also differentially expressed in tumors, e.g., ovarian tumors, relative to their expression in normal tissue samples.

“Differential expression”, as used herein, includes both quantitative as well as qualitative differences in the temporal and/or tissue expression pattern of a gene. Thus, a differentially expressed gene may have its expression activated or inactivated in normal versus cardiovascular or tumorigenic disease conditions (for example, in an experimental cardiovascular or tumorigenic disease system). The degree to which expression differs in normal versus cardiovascular or tumorigenic disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic cardiovascular or tumorigenic disease evaluation, or may be used in methods for identifying compounds useful for the treatment of cardiovascular or tumorigenic disease. In addition, a differentially expressed gene involved in cardiovascular or tumorigenic disease may represent a target gene such that modulation of the level of target gene expression or of target gene product activity may act to ameliorate a cardiovascular or tumorigenic disease condition. Compounds that modulate target gene expression or activity of the target gene product can be used in the treatment of cardiovascular or tumorigenic disease. Although the GPCR 4941 genes described herein may be differentially expressed with respect to cardiovascular or tumorigenic disease, and/or their products may interact with gene products important to cardiovascular or tumorigenic disease, the genes may also be involved in mechanisms important to additional cardiovascular, endothelial cell, and tumor cell processes.

The GPCR 4941 molecules of the present invention may be signal transduction proteins that function to modulate cell proliferation, differentiation, and motility. Thus, the GPCR 4941 molecules of the present invention may play a role in cellular growth signaling mechanisms. As used herein, the term “cellular growth signaling mechanisms” includes signal transmission from cell receptors, e.g., G protein coupled receptors, which regulates 1) cell transversal through the cell cycle, 2) cell differentiation, 3) cell survival, and/or 4) cell migration and patterning.

Accordingly, the GPCR 4941 molecules of the present invention may be involved in cellular signal transduction pathways that modulate endothelial cell activity. As used herein, an “endothelial cell activity” or “endothelial cell function” includes cell proliferation differentiation, migration, and expression of cell surface adhesion molecules and/or genes associated with angiogenesis (e.g., TIE-2, FLT and FLK), as well as cellular processes that contribute to the physiological role of endothelial cells (e.g., vascularization and the regulation of blood flow). The GPCR 4941 molecules of the present invention may also be involved in cellular signal transduction pathways that modulate tumor cell activity. As used herein, a “tumor cell activity” or “tumor cell function” includes cell proliferation transformation, migration, anchorage independent growth, and expression of genes associated with tumorigenesis (e.g., oncogenes or tumor suppressor genes). Thus, the GPCR 4941 molecules, by participating in cellular signal transduction pathways, may modulate cell behavior and act as targets and therapeutic agents for controlling cellular proliferation, differentiation, and migration, as well as angiogenesis and vascularization.

The GPCR 4941 molecules of the present invention may also act as novel diagnostic targets and therapeutic agents for cardiovascular or tumorigenic diseases or disorders, as well as disorders associated with the human chromosome 2q21-q22 locus (McKee et al. (1997) Genomics, 46:426-434). As used herein, “cardiovascular disease” or a “cardiovascular disorder” includes a disease or disorder which affects the cardiovascular system, e.g., the heart or the blood vessels. A cardiovascular disorder includes disorders such as arteriosclerosis, ischemia reperfusion injury, restenosis, arterial inflammation, vascular wall remodeling, yentricular remodeling, rapid ventricular pacing, coronary microembolism, tachycardia, bradycardia, pressure overload, aortic bending, coronary artery ligation, vascular heart disease, atrial fibrillation, long-QT syndrome, congestive heart failure, sinus node dysfunction, angina, heart failure, hypertension, atrial fibrillation, atrial flutter, dilated cardiomyopathy, idiopathic cardiomyopathy, myocardial infarction, coronary artery disease, coronary artery spasm, ischemic disease, arrhythmia, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovasular disease or disorder also includes an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

As used herein, a “tumorigenic disease or disorder” includes a disease or disorder characterized by aberrantly regulated cell growth, proliferation, differentiation, adhesion, or migration, resulting in the production of or tendency to produce tumors. As used herein, a “tumor” includes a normal benign or malignant mass of tissue. Examples of tumorigenic diseases include cancer, e.g., carcinoma, sarcoma, lymphoma or leukemia, examples of which include, but are not limited to, ovarian, lung, breast, endometrial, uterine, hepatic, gastrointestinal, prostate, colorectal, and brain cancer.

The present invention provides methods for identifying the presence of a GPCR 4941 nucleic acid or polypeptide molecule associated with a cardiovascular disorder, an endothelial cell disorder or a tumorigenic disorder. In addition, the invention provides methods for identifying a subject at risk for a cardiovascular disorder, an endothelial cell disorder or a tumorigenic disorder, by detecting the presence of a GPCR 4941 nucleic acid or polypeptide molecule.

The invention also provides a method for identifying a compound capable of treating a cardiovascular disorder, an endothelial cell disorder or a tumorigenic disorder, characterized by aberrant GPCR 4941 nucleic acid expression or GPCR 4941 protein activity by assaying the ability of the compound to modulate the expression of a GPCR 4941 nucleic acid or the activity of a GPCR 4941 protein. Furthermore, the invention provides a method for treating a subject having a cardiovascular disorder, an endothelial cell disorder, or a tumorigenic disorder characterized by aberrant GPCR 4941 protein activity or aberrant GPCR 4941 nucleic acid expression by administering to the subject a GPCR 4941 modulator which is capable of modulating GPCR 4941 protein activity or GPCR 4941 nucleic acid expression.

Moreover, the invention provides a method for identifying a compound capable of modulating an endothelial cell activity or a tumor cell activity by modulating the expression of a GPCR 4941 nucleic acid or the activity of a GPCR 4941 protein. The invention provides a method for modulating an endothelial cell activity or a tumor cell activity comprising contacting an endothelial cell with a GPCR 4941 modulator.

Various aspects of the invention are described in further detail in the following subsections.

1. Screening Assays

The invention provides a method (also referred to herein as a “screening assay”) for identifying modulators, i.e., candidate or test compounds or agents (e.g., peptides, peptidomimetics, small molecules (organic or inorganic) or other drugs) which bind to GPCR 4941 proteins, have a stimulatory or inhibitory effect on, for example, GPCR 4941 expression or GPCR 4941 activity, or have a stimulatory or inhibitory effect on, for example, the expression or activity of a GPCR 4941 substrate.

These assays are designed to identify compounds that bind to a GPCR 4941 protein, bind to other intracellular or extracellular proteins that interact with a GPCR 4941 protein, and interfere with the interaction of the GPCR 4941 protein with other cellular or extracellular proteins. For example, in the case of the GPCR 4941 protein, which is a transmembrane receptor-type protein, such techniques can identify ligands for such a receptor. A GPCR 4941 protein ligand can, for example, be used to ameliorate cardiovascular diseases, e.g., atherosclerosis, ischemia/reperfusion, hypertension, restenosis, and arterial inflammation; endothelial cell disorders, such as disorders associated with aberrant endothelial cell growth, angiogenesis and/or vascularization; and tumorigenic diseases, e.g., ovarian tumors. Such compounds may include, but are not limited to peptides, antibodies, or small organic or inorganic compounds. Such compounds may also include other cellular proteins.

Compounds identified via assays such as those described herein may be useful, for example, for ameliorating cardiovascular or tumorigenic disease. In instances whereby a cardiovascular or tumorigenic disease condition results from an overall lower level of GPCR 4941 gene expression and/or GPCR 4941 protein in a cell or tissue, compounds that interact with the GPCR 4941 protein may include compounds which accentuate or amplify the activity of the bound GPCR 4941 protein. Such compounds would bring about an effective increase in the level of GPCR 4941 protein activity, thus ameliorating symptoms.

In other instances, mutations within the GPCR 4941 gene may cause aberrant types or excessive amounts of GPCR 4941 proteins to be made which have a deleterious effect that leads to a cardiovascular or tumorigenic disease. Similarly, physiological conditions may cause an excessive increase in GPCR 4941 gene expression leading to a cardiovascular or tumorigenic disease. In such cases, compounds that bind to a GPCR 4941 protein may be identified that inhibit the activity of the GPCR 4941 protein. Assays for testing the effectiveness of compounds identified by techniques such as those described in this section are discussed herein.

In one embodiment, the invention provides assays for screening candidate or test compounds which are substrates of a GPCR 4941 protein or polypeptide or biologically active portion thereof. In another embodiment, the invention provides assays for screening candidate or test compounds which bind to or modulate the activity of a GPCR 4941 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to-peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a GPCR 4941 protein or biologically active portion thereof is contacted with a test compound and the ability of the test compound to modulate GPCR 4941 activity is determined. Determining the ability of the test compound to modulate GPCR 4941 activity can be accomplished by monitoring, for example, intracellular calcium, IP3, cAMP, or diacylglycerol concentration, the phosphorylation profile of intracellular proteins, cell proliferation and/or migration, gene expression of, for example, cell surface adhesion molecules or genes associated with angiogenesis or tumorigenesis, or the activity of a GPCR 4941-regulated transcription factor. The cell can be of mammalian origin, e.g., an endothelial cell or an ovarian cell such as a cell derived from the surface epithelium or ascites fluid. In one embodiment, compounds that interact with a GPCR 4941 receptor domain can be screened for their ability to function as ligands, i.e., to bind to the GPCR 4941 receptor and modulate a signal transduction pathway. Identification of GPCR 4941 ligands, and measuring the activity of the ligand-receptor complex, leads to the identification of modulators (e.g., antagonists) of this interaction. Such modulators may be useful in the treatment of cardiovascular or tumorigenic disease.

The ability of the test compound to modulate GPCR 4941 binding to a substrate or to bind to GPCR 4941 can also be determined. Determining the ability of the test compound to modulate GPCR 4941 binding to a substrate can be accomplished, for example, by coupling the GPCR 4941 substrate with a radioisotope or enzymatic label such that binding of the GPCR 4941 substrate to GPCR 4941 can be determined by detecting the labeled GPCR 4941 substrate in a complex. GPCR 4941 could also be coupled with a radioisotope or enzymatic label to monitor the ability of a test compound to modulate GPCR 4941 binding to a GPCR 4941 substrate in a complex. Determining the ability of the test compound to bind GPCR 4941 can be accomplished, for example, by coupling the compound with a radioisotope or enzymatic label such that binding of the compound to GPCR 4941 can be determined by detecting the labeled GPCR 4941 compound in a complex. For example, compounds (e.g., GPCR 4941 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a GPCR 4941 ligand or substrate) to interact with GPCR 4941 without the labeling of any of the interactants. For example, a microphysiometer can be used to detect the interaction of a compound with GPCR 4941 without the labeling of either the compound or the GPCR 4941 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and GPCR 4941.

In another embodiment, an assay is a cell-based assay comprising contacting a cell expressing a GPCR 4941 target molecule (e.g., a GPCR 4941 substrate) with a test compound and determining the ability of the test compound to modulate (e.g., stimulate or inhibit) the activity of the GPCR 4941 target molecule. Determining the ability of the test compound to modulate the activity of a GPCR 4941 target molecule can be accomplished, for example, by determining the ability of the GPCR 4941 protein to bind to or interact with the GPCR 4941 target molecule.

Determining the ability of the GPCR 4941 protein or a biologically active fragment thereof, to bind to or interact with a GPCR 4941 target molecule can be accomplished by one of the methods described above for determining direct binding. In a preferred embodiment, determining the ability of the GPCR 4941 protein to bind to or interact with a GPCR 4941 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression, cell proliferation or migration).

In yet another embodiment, an assay of the present invention is a cell-free assay in which a GPCR 4941 protein or biologically active portion thereof, is contacted with a test compound and the ability of the test compound to bind to the GPCR 4941 protein or biologically active portion thereof is determined. Preferred biologically active portions of the GPCR 4941 proteins to be used in assays of the present invention include fragments which participate in interactions with non-GPCR 4941 molecules, e.g., fragments with high surface probability scores. Binding of the test compound to the GPCR 4941 protein can be determined either directly or indirectly as described above. In a preferred embodiment, the assay includes contacting the GPCR 4941 protein or biologically active portion thereof with a known compound which binds GPCR 4941 to form an assay mixture, contacting the assay mixture with a test compound, and determining the ability of the test compound to interact with a GPCR 4941 protein, wherein determining the ability of the test compound to interact with a GPCR 4941 protein comprises determining the ability of the test compound to preferentially bind to GPCR 4941 or biologically active portion thereof as compared to the known compound. Compounds that modulate the interaction of GPCR 4941 with a known target protein may be useful in regulating the activity of a GPCR 4941 protein, especially a mutant GPCR 4941 protein.

In another embodiment, the assay is a cell-free assay in which a GPCR 4941 protein or biologically active portion thereof is contacted with a test compound and the ability of the test compound to modulate (e.g., stimulate or inhibit) the activity of the GPCR 4941 protein or biologically active portion thereof is determined. Determining the ability of the test compound to modulate the activity of a GPCR 4941 protein can be accomplished, for example, by determining the ability of the GPCR 4941 protein to bind to a GPCR 4941 target molecule by one of the methods described above for determining direct binding. Determining the ability of the GPCR 4941 protein to bind to a GPCR 4941 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a GPCR 4941 protein can be accomplished by determining the ability of the GPCR 4941 protein to further modulate the activity of a downstream effector of a GPCR 4941 target molecule. For example, the activity of the effector molecule on an appropriate target can be determined or the binding of the effector to an appropriate target can be determined as previously described.

In yet another embodiment, the cell-free assay involves contacting a GPCR 4941 protein or biologically active portion thereof with a known compound which binds the GPCR 4941 protein to form an assay mixture, contacting the assay mixture with a test compound, and determining the ability of the test compound to interact with the GPCR 4941 protein, wherein determining the ability of the test compound to interact with the GPCR 4941 protein comprises determining the ability of the GPCR 4941 protein to preferentially bind to or modulate the activity of a GPCR 4941 target molecule.

In more than one embodiment of the above assay methods of the present invention, it may be desirable to immobilize either GPCR 4941 or its target molecule to facilitate separation of complexed from uncomplexed forms of one or both of the proteins, as well as to accommodate automation of the assay. Binding of a test compound to a GPCR 4941 protein, or interaction of a GPCR 4941 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/GPCR 4941 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or GPCR 4941 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of GPCR 4941 binding or activity determined using standard techniques.

Other techniques for immobilizing proteins on matrices can also be used in the screening assays of the invention. For example, either a GPCR 4941 protein or a GPCR 4941 target molecule can be immobilized utilizing conjugation of biotin and streptavidin. Biotinylated GPCR 4941 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with GPCR 4941 protein or target molecules but which do not interfere with binding of the GPCR 4941 protein to its target molecule can be derivatized to the wells of the plate, and unbound target or GPCR 4941 protein trapped in the wells by antibody conjugation. Methods for detecting such complexes, in addition to those described above for the GST-immobilized complexes, include immunodetection of complexes using antibodies reactive with the GPCR 4941 protein or target molecule, as well as enzyme-linked assays which rely on detecting an enzymatic activity associated with the GPCR 4941 protein or target molecule.

In another embodiment, modulators of GPCR 4941 expression are identified in a method wherein a cell is contacted with a candidate compound and the expression of GPCR 4941 mRNA or protein in the cell is determined. The level of expression of GPCR 4941 mRNA or protein in the presence of the candidate compound is compared to the level of expression of GPCR 4941 mRNA or protein in the absence of the candidate compound. The candidate compound can then be identified as a modulator of GPCR 4941 expression based on this comparison. For example, when expression of GPCR 4941 mRNA or protein is greater (statistically significantly greater) in the presence of the candidate compound than in its absence, the candidate compound is identified as a stimulator of GPCR 4941 mRNA or protein expression. Alternatively, when expression of GPCR 4941 mRNA or protein is less (statistically significantly less) in the presence of the candidate compound than in its absence, the candidate compound is identified as an inhibitor of GPCR 4941 mRNA or protein expression. The level of GPCR 4941 mRNA or protein expression in the cells can be determined by methods described herein for detecting GPCR 4941 mRNA or protein.

In yet another aspect of the invention, the GPCR 4941 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with GPCR 4941 (“GPCR 4941-binding proteins” or “GPCR 4941-bp”) and are involved in GPCR 4941 activity. Such GPCR 4941-binding proteins are also likely to be involved in the propagation of signals by the GPCR 4941 proteins or GPCR 4941 targets as, for example, downstream elements of a GPCR 4941-mediated signaling pathway. Alternatively, such GPCR 4941-binding proteins are likely to be GPCR 4941 inhibitors.

The two-hybrid system is based on the modular nature of most transcription factors, which consist of separable DNA-binding and activation domains. Briefly, the assay utilizes two different DNA constructs. In one construct, the gene that codes for a GPCR 4941 protein is fused to a gene encoding the DNA binding domain of a known transcription factor (e.g., GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a GPCR 4941-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the GPCR 4941 protein.

In another aspect, the invention pertains to a combination of two or more of the assays described herein. For example, a modulating agent can be identified using a cell-based or a cell free assay, and the ability of the agent to modulate the activity of a GPCR 4941 protein can be confirmed in vivo, e.g., in an animal such as an animal model for cardiovascular or tumorigenic disease, as described herein.

This invention further pertains to novel agents identified by the above-described screening assays. Accordingly, it is within the scope of this invention to further use an agent identified as described herein in an appropriate animal model. For example, an agent identified as described herein (e.g., a GPCR 4941 modulating agent, an antisense GPCR 4941 nucleic acid molecule, a GPCR 4941-specific antibody, or a GPCR 4941-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate cardiovascular or tumorigenic disease symptoms. Cell-based and animal model-based assays for the identification of compounds exhibiting such an ability to ameliorate cardiovascular or tumorigenic disease systems are described herein.

In one aspect, cell-based systems, as described herein, may be used to identify compounds which may act to ameliorate cardiovascular or tumorigenic disease symptoms. For example, such cell systems may be exposed to a compound, suspected of exhibiting an ability to ameliorate cardiovascular or tumorigenic disease symptoms, at a sufficient concentration and for a time sufficient to elicit such an amelioration of cardiovascular or tumorigenic disease symptoms in the exposed cells. After exposure, the cells are examined to determine whether one or more of the cardiovascular or tumorigenic disease cellular phenotypes has been altered to resemble a more normal or more wild type, non-cardiovascular disease or non-tumorigenic disease phenotype. Cellular phenotypes that are associated with cardiovascular disease states include aberrant proliferation and migration, angiogenesis, deposition of extracellular matrix components, accumulation of intracellular lipids, and expression of growth factors, cytokines, and other inflammatory mediators. Cellular phenotypes that are associated with tumorigenic disease states include aberrant proliferation and migration, angiogenesis, anchorage independent growth, and loss of contact inhibition.

In addition, animal-based cardiovascular disease systems, such as those described herein, may be used to identify compounds capable of ameliorating cardiovascular or tumorigenic disease symptoms. Such animal models may be used as test substrates for the identification of drugs, pharmaceuticals, therapies, and interventions which may be effective in treating cardiovascular or tumorigenic disease. For example, animal models may be exposed to a compound, suspected of exhibiting an ability to ameliorate cardiovascular or tumorigenic disease symptoms, at a sufficient concentration and for a time sufficient to elicit such an amelioration of cardiovascular or tumorigenic disease symptoms in the exposed animals. The response of the animals to the exposure may be monitored by assessing the reversal of disorders associated with cardiovascular disease, for example, by counting the number of atherosclerotic plaques and/or measuring their size before and after treatment. In addition, the animals may be monitored by assessing the reversal of disorders associated with tumorigenic disease, for example, reduction in tumor burden, tumor size, and invasive and/or metastatic potential before and after treatment.

With regard to intervention, any treatments which reverse any aspect of cardiovascular or tumorigenic disease symptoms should be considered as candidates for human cardiovascular or tumorigenic disease therapeutic intervention. Dosages of test agents may be determined by deriving dose-response curves.

Additionally, gene expression patterns may be utilized to assess the ability of a compound to ameliorate cardiovascular or tumorigenic disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, atherosclerosis, ischemia/reperfusion, hypertension, restenosis, and arterial inflammation, including any of the control or experimental conditions described herein, for example, endothelial cells treated under conditions of laminar sheer stress (LSS), cytokine stimulation, growth on Matrigel, and proliferation. Other conditions may include, for example, cell proliferation, differentiation, transformation, tumorigenesis, metastasis, and carcinogen exposure. Gene expression profiles may be generated, for example, by utilizing a differential display procedure, Northern analysis and/or RT-PCR. In one embodiment, GPCR 4941 gene sequences may be used as probes and/or PCR primers for the generation and corroboration of such gene expression profiles.

Gene expression profiles may be characterized for known states, either cardiovascular or tumorigenic disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cardiovascular or tumorigenic disease model system to more closely resemble the control system. Administration of a compound may, alternatively, cause the gene expression profile of a control system to begin to mimic a cardiovascular or tumorigenic disease state. Such a compound may, for example, be used in further characterizing the compound of interest, or may be used in the generation of additional animal models.

2. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining GPCR 4941 protein and/or nucleic acid expression as well as GPCR 4941 activity, in the context of a biological sample (e.g., blood, serum, cells, tissue) to thereby determine whether an individual is afflicted with a disease or disorder, or is at risk of developing a cardiovascular disorder, an endothelial cell disorder, or a tumorigenic disorder, associated with aberrant or unwanted GPCR 4941 expression or activity. The invention also provides for prognostic (or predictive) assays for determining whether an individual is at risk of developing a disorder associated with GPCR 4941 protein, nucleic acid expression or activity. For example, mutations in a GPCR 4941 gene can be assayed in a biological sample. Such assays can be used for prognostic or predictive purpose to thereby prophylactically treat an individual prior to the onset of a disorder characterized by or associated with GPCR 4941 protein, nucleic acid expression or activity.

Another aspect of the invention pertains to monitoring the influence of agents (e.g., drugs, compounds) on the expression or activity of GPCR 4941 in clinical trials.

These and other agents are described in further detail in the following sections.

A. Diagnostic Assays

The present invention encompasses methods for diagnostic and prognostic evaluation of cardiovascular or tumorigenic disease conditions, and for the identification of subjects exhibiting a predisposition to such conditions.

An exemplary method for detecting the presence or absence of GPCR 4941 protein or nucleic acid in a biological sample involves obtaining a biological sample from a test subject and contacting the biological sample with a compound or an agent capable of detecting GPCR 4941 protein or nucleic acid (e.g., mRNA, or genomic DNA) that encodes GPCR 4941 protein such that the presence of GPCR 4941 protein or nucleic acid is detected in the biological sample. A preferred agent for detecting GPCR 4941 mRNA or genomic DNA is a labeled nucleic acid probe capable of hybridizing to GPCR 4941 mRNA or genomic DNA. The nucleic acid probe can be, for example, the GPCR 4941 nucleic acid set forth in SEQ ID NO:1 or 3, or a portion thereof, such as an oligonucleotide of at least 15, 20, 25, 30, 35, 40, 45, 50, 100, 250 or 500 nucleotides in length and sufficient to specifically hybridize under stringent conditions to GPCR 4941 mRNA or genomic DNA. Other suitable probes for use in the diagnostic assays of the invention are described herein.

A preferred agent for detecting GPCR 4941 protein is an antibody capable of binding to GPCR 4941 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect GPCR 4941 mRNA, protein, or genomic DNA in a biological sample in vitro as well as in vivo. For example, in vitro techniques for detection of GPCR 4941 mRNA include Northern hybridizations and in situ hybridizations. In vitro techniques for detection of GPCR 4941 protein include enzyme linked immunosorbent assays (ELISAs), Western blots, immunoprecipitations and immunofluorescence. In vitro techniques for detection of GPCR 4941 genomic DNA include Southern hybridizations. Furthermore, in vivo techniques for detection of GPCR 4941 protein include introducing into a subject a labeled anti-GPCR 4941 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting GPCR 4941 protein, mRNA, or genomic DNA, such that the presence of GPCR 4941 protein, mRNA or genomic DNA is detected in the biological sample, and comparing the presence of GPCR 4941 protein, mRNA or genomic DNA in the control sample with the presence of GPCR 4941 protein, mRNA or genomic DNA in the test sample.

The invention also encompasses kits for detecting the presence of GPCR 4941 in a biological sample. For example, the kit can comprise a labeled compound or agent capable of detecting GPCR 4941 protein or mRNA in a biological sample; means for determining the amount of GPCR 4941 in the sample; and means for comparing the amount of GPCR 4941 in the sample with a standard. The compound or agent can be packaged in a suitable container. The kit can further comprise instructions for using the kit to detect GPCR 4941 protein or nucleic acid.

B. Prognostic Assays

The diagnostic methods described herein can furthermore be utilized to identify subjects having or at risk of developing a cardiovascular or tumorigenic disease or disorder associated with aberrant or unwanted GPCR 4941 expression or activity. As used herein, the term “aberrant” includes a GPCR 4941 expression or activity which deviates from the wild type GPCR 4941 expression or activity. Aberrant expression or activity includes increased or decreased expression or activity, as well as expression or activity which does not follow the wild type developmental pattern of expression or the subcellular pattern of expression. For example, aberrant GPCR 4941 expression or activity is intended to include the cases in which a mutation in the GPCR 4941 gene causes the GPCR 4941 gene to be under-expressed or over-expressed and situations in which such mutations result in a non-functional GPCR 4941 protein or a protein which does not function in a wild-type fashion, e.g., a protein which does not interact with a GPCR 4941 ligand or substrate, or one which interacts with a non-GPCR 4941 ligand or substrate. As used herein, the term “unwanted” includes an unwanted phenomenon involved in a biological response such as cellular proliferation. For example, the term unwanted includes a GPCR 4941 expression pattern or a GPCR 4941 protein activity which is undesirable in a subject.

The assays described herein, such as the preceding diagnostic assays or the following assays, can be utilized to identify a subject having or at risk of developing a disorder associated with a misregulation in GPCR 4941 protein activity or nucleic acid expression, such as a cardiovascular disorder, an endothelial cell disorder, or a tumorigenic disorder. Alternatively, the prognostic assays can be utilized to identify a subject having or at risk for developing a cardiovascular disorder, an endothelial cell disorder, or a tumorigenic disorder, associated with a misregulation in GPCR 4941 protein activity or nucleic acid expression. Thus, the present invention provides a method for identifying a disease or disorder associated with aberrant or unwanted GPCR 4941 expression or activity in which a test sample is obtained from a subject and GPCR 4941 protein or nucleic acid (e.g., mRNA or genomic DNA) is detected, wherein the presence of GPCR 4941 protein or nucleic acid is diagnostic for a subject having or at risk of developing a disease or disorder associated with aberrant or unwanted GPCR 4941 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted GPCR 4941 expression or activity. For example, such methods can be used to determine whether a subject can be effectively treated with an agent for a cardiovascular disorder, an endothelial cell disorder, or a tumorigenic disorder. Thus, the present invention provides methods for determining whether a subject can be effectively treated with an agent for a cardiovascular disorder, an endothelial cell disorder, or a tumorigenic disorder, associated with aberrant or unwanted GPCR 4941 expression or activity in which a test sample is obtained and GPCR 4941 protein or nucleic acid expression or activity is detected (e.g., wherein the abundance of GPCR 4941 protein or nucleic acid expression or activity is diagnostic for a subject that can be administered the agent to treat a disorder associated with aberrant or unwanted GPCR 4941 expression or activity).

The methods of the invention can also be used to detect genetic alterations in a GPCR 49.41 gene, thereby determining if a subject with the altered gene is at risk for a disorder characterized by misregulation in GPCR 4941 protein activity or nucleic acid expression, such as a cardiovascular disorder, an endothelial cell disorder, or a tumorigenic disorder. In preferred embodiments, the methods include detecting, in a sample of cells from the subject, the presence or absence of a genetic alteration characterized by at least one of an alteration affecting the integrity of a gene encoding a GPCR 4941-protein, or the misexpression of the GPCR 4941 gene. For example, such genetic alterations can be detected by ascertaining the existence of at least one of 1) a deletion of one or more nucleotides from a GPCR 4941 gene; 2) an addition of one or more nucleotides to a GPCR 4941 gene; 3) a substitution of one or more nucleotides of a GPCR 4941 gene, 4) a chromosomal rearrangement of a GPCR 4941 gene; 5) an alteration in the level of a messenger RNA transcript of a GPCR 4941 gene, 6) aberrant modification of a GPCR 4941 gene, such as of the methylation pattern of the genomic DNA, 7) the presence of a non-wild type splicing pattern of a messenger RNA transcript of a GPCR 4941 gene, 8) a non-wild type level of a GPCR 4941-protein, 9) allelic loss of a GPCR 4941 gene, and 10) inappropriate post-translational modification of a GPCR 4941-protein. As described herein, there are a large number of assays known in the art which can be used for detecting alterations in a GPCR 4941 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the GPCR 4941-gene (see Abravaya et al. (1995) Nucleic Acids Res 0.23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a GPCR 4941 gene under conditions such that hybridization and amplification of the GPCR 4941-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Other amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a GPCR 4941 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in GPCR 4941 can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in GPCR 4941 can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GPCR 4941 gene and detect mutations by comparing the sequence of the sample GPCR 4941 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36: 127-162; and Griffin et al. (1993) Appl. Biochem. Biotechnol. 38:147-159).

Other methods for detecting mutations in the GPCR 4941 gene include methods in which protection-from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type GPCR 4941 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GPCR 4941 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a GPCR 4941 sequence, e.g., a wild-type GPCR 4941 sequence, is hybridized to a cDNA or other DNA product from a test cell(s). The duplex is treated with a DNA mismatch repair enzyme, and the cleavage products, if any, can be detected from electrophoresis protocols or the like (described in, for example, U.S. Pat. No. 5,459,039).

In other embodiments, alterations in electrophoretic mobility will be used to identify mutations in GPCR 4941 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control GPCR 4941 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a GPCR 4941 gene.

Furthermore, any cell type or tissue in which GPCR 4941 is expressed may be utilized in the prognostic assays described herein.

C. Monitoring of Effects During Clinical Trials

The present invention provides methods for evaluating the efficacy of drugs and monitoring the progress of patients involved in clinical trials for the treatment of cardiovascular or tumorigenic disease.

Monitoring the influence of agents (e.g., drugs) on the expression or activity of a GPCR 4941 protein (e.g., the modulation of cell proliferation and/or migration) can be applied not only in basic drug screening, but also in clinical trials. For example, the effectiveness of an agent determined by a screening assay as described herein to increase GPCR 4941 gene expression, protein levels, or upregulate GPCR 4941 activity, can be monitored in clinical trials of subjects exhibiting decreased GPCR 4941 gene expression, protein levels, or downregulated GPCR 4941 activity. Alternatively, the effectiveness of an agent determined by a screening assay to decrease GPCR 4941 gene expression, protein levels, or downregulate GPCR 4941 activity, can be monitored in clinical trials of subjects exhibiting increased GPCR 4941 gene expression, protein levels, or upregulated GPCR 4941 activity. In such clinical trials, the expression or activity of a GPCR 4941 gene, and preferably, other genes that have been implicated in, for example, a GPCR 4941-associated disorder can be used as a “read out” or markers of the phenotype a particular cell, e.g., an endothelial cell or an ovarian cell. In addition, the expression of a GPCR 4941 gene, or the level of GPCR 4941 protein activity may be used as a read out of a particular drug or agent's effect on a cardiovascular or tumorigenic disease state.

For example, and not by way of limitation, genes, including GPCR 4941, that are modulated in cells by treatment with an agent (e.g., compound, drug or small molecule) which modulates GPCR 4941 activity (e.g., identified in a screening assay as described herein) can be identified. Thus, to study the effect of agents on GPCR 4941-associated disorders (e.g., cardiovascular disorders, endothelial cell disorders, or tumorigenic disorders), for example, in a clinical trial, cells can be isolated and RNA prepared and analyzed for the levels of expression of GPCR 4941 and other genes implicated in the GPCR 4941-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of GPCR 4941 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent;(ii) detecting the level of expression of a GPCR 4941 protein, mRNA, or genomic DNA in the preadministration sample; (iii) obtaining one or more post-administration samples from the subject; (iv) detecting the level of expression or activity of the GPCR 4941 protein, mRNA, or genomic DNA in the post-administration samples; (v) comparing the level of expression or activity of the GPCR 4941 protein, mRNA, or genomic DNA in the pre-administration sample with the GPCR 4941 protein, mRNA, or genomic DNA in the post administration sample or samples; and (vi) altering the administration of the agent to the subject accordingly. For example, increased administration of the agent may be desirable to increase the expression or activity of GPCR 4941 to higher levels than detected, i.e., to increase the effectiveness of the agent. Alternatively, decreased administration of the agent may be desirable to decrease expression or activity of GPCR 4941 to lower levels than detected, i.e. to decrease the effectiveness of the agent. According to such an embodiment, GPCR 4941 expression or activity may be used as an indicator of the effectiveness of an agent, even in the absence of an observable phenotypic response.

3. Methods of Treatment:

The present invention provides for both prophylactic and therapeutic methods of treating a subject at risk of (or susceptible to) a disorder or having a disorder associated with aberrant or unwanted GPCR 4941 expression or activity, e.g. a cardiovascular disorder, an endothelial cell disorder, or a tumorigenic disorder. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the GPCR 4941 molecules of the present invention or GPCR 4941 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a cardiovascular disease or condition, or a tumorigenic disorder associated with an aberrant or unwanted GPCR 4941 expression or activity, by administering to the subject a GPCR 4941 or an agent which modulates GPCR 4941 expression or at least one GPCR 4941 activity. Subjects at risk for a cardiovascular disorder, an endothelial cell disorder or a tumorigenic disorder which is caused or contributed to by aberrant or unwanted GPCR 4941 expression or activity can be identified by, for example, any or a combination of diagnostic or prognostic assays as described herein. Administration of a prophylactic agent can occur prior to the manifestation of symptoms characteristic of the GPCR 4941 aberrancy, such that a disease or disorder is prevented or, alternatively, delayed in its progression. Depending on the type of GPCR 4941 aberrancy, for example, a GPCR 4941, GPCR 4941 agonist or GPCR 4941 antagonist agent can be used for treating the subject. The appropriate agent can be determined based on screening assays described herein.

B. Therapeutic Methods

Described herein are methods and compositions whereby cardiovascular or tumorigenic disease symptoms may be ameliorated. Certain cardiovascular or tumorigenic diseases are brought about, at least in part, by an excessive level of a gene product, or by the presence of a gene product exhibiting an abnormal or excessive activity. As such, the reduction in the level and/or activity of such gene products would bring about the amelioration of cardiovascular or tumorigenic disease symptoms. Techniques for the reduction of gene expression levels or the activity of a protein are discussed below.

Alternatively, certain other cardiovascular or tumorigenic diseases are brought about, at least in part, by the absence or reduction of the level of gene expression, or a reduction in the level of a protein's activity. As such, an increase in the level of gene expression and/or the activity of such proteins would bring about the amelioration of cardiovascular or tumorigenic disease symptoms.

In some cases, the up-regulation of a gene in a disease state reflects a protective role for that gene product in responding to the disease condition. Enhancement of such a gene's expression, or the activity of the gene product, will reinforce the protective effect it exerts. Some cardiovascular or tumorigenic disease states may result from an abnormally low level of activity of such a protective gene. In these cases also, an increase in the level of gene expression and/or the activity of such gene products would bring about the amelioration of cardiovascular or tumorigenic disease symptoms. Techniques for increasing target gene expression levels or target gene product activity levels are discussed herein.

Accordingly, another aspect of the invention pertains to methods of modulating GPCR 4941 expression or activity for therapeutic purposes. Accordingly, in an exemplary embodiment, the modulatory method of the invention involves contacting a cell with a GPCR 4941 or agent that modulates one or more of the activities of GPCR 4941 protein activity associated with the cell (e.g., an endothelial cell or an ovarian cell). An agent that modulates GPCR 4941 protein activity can be an agent as described herein, such as a nucleic acid or a protein, a naturally-occurring target molecule of a GPCR 4941 protein (e.g., a GPCR 4941 ligand or substrate), a GPCR 4941 antibody, a GPCR 4941 agonist or antagonist, a peptidomimetic of a GPCR 4941 agonist or antagonist, or other small molecule. In one embodiment, the agent stimulates one or more GPCR 4941 activities. Examples of such stimulatory agents include active GPCR 4941 protein and a nucleic acid molecule encoding GPCR 4941 that has been introduced into the cell. In another embodiment, the agent inhibits one or more GPCR 4941 activities. Examples of such inhibitory agents include antisense GPCR 4941 nucleic acid molecules, anti-GPCR 4941 antibodies, and GPCR 4941 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a GPCR 4941 protein or nucleic acid molecule. In one embodiment, the method involves administering an agent (e.g., an agent identified by a screening assay described herein), or combination of agents that modulates (e.g., upregulates or downregulates) GPCR 4941 expression or activity. In another embodiment, the method involves administering a GPCR 4941 protein or nucleic acid molecule as therapy to compensate for reduced, aberrant, or unwanted GPCR 4941 expression or activity.

Stimulation of GPCR 4941 activity is desirable in situations in which GPCR 4941 is abnormally downregulated and/or in which increased GPCR 4941 activity is likely to have a beneficial effect. Likewise, inhibition of GPCR 4941 activity is desirable in situations in which GPCR 4941 is abnormally upregulated and/or in which decreased GPCR 4941 activity is likely to have a beneficial effect.

(i) Methods for Inhibiting Target Gene Expression, Synthesis. or Activity

As discussed above, genes involved in cardiovascular disorders, endothelial cell disorders (e.g., disorders associated with aberrant endothelial cell growth, migration, angiogenesis and/or vascularization) or tumorigenic disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate cardiovascular or tumorigenic disease symptoms. Such molecules may include, but are not limited to, small organic molecules, peptides, antibodies, and the like.

For example, compounds can be administered that compete with endogenous ligand for the GPCR 4941 protein. The resulting reduction in the amount of ligand-bound GPCR 4941 protein will modulate endothelial cell physiology. Compounds that can be particularly useful for this purpose include, for example, soluble proteins or peptides, such as peptides comprising one or more of the extracellular domains, or portions and/or analogs thereof, of the GPCR 4941 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the GPCR 4941 receptor site, but do not activate the protein, (e.g., receptor-ligand antagonists) can be effective in inhibiting GPCR 4941 protein activity.

Further, antisense and ribozyme molecules which inhibit expression of the GPCR 4941 gene may also be used in accordance with the invention to inhibit aberrant GPCR 4941 gene activity. Still further, triple helix molecules may be utilized in inhibiting aberrant GPCR 4941 gene activity.

The antisense nucleic acid molecules of the invention are typically administered to a subject or generated in situ such that they hybridize with or bind to cellular mRNA and/or genomic DNA encoding a GPCR 4941 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specifics double-stranded hybrids with complementary RNA in which, contrary to the usual β-units; the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave GPCR 4941 mRNA transcripts to thereby inhibit translation of GPCR 4941 mRNA. A ribozyme having specificity for a GPCR 4941-encoding nucleic acid can be designed based upon the nucleotide sequence of a GPCR 4941 cDNA disclosed herein (i.e., SEQ ID NO:1 or 3). For example, a derivative of a Tetrahymena L-19 IVS RNA can be constructed in which the nucleotide sequence of the active site is complementary to the nucleotide sequence to be cleaved in a GPCR 4941-encoding mRNA (see, for example, Cech et al. U.S. Pat. No. 4,987,071; and Cech et al. U.S. Pat. No. 5,116,742). Alternatively, GPCR 4941 mRNA can be used to select a catalytic RNA having a specific ribonuclease activity from a pool of RNA molecules (see, for example, Bartel, D. and Szostak, J. W. (1993) Science 261:1411-1418).

GPCR 4941 gene expression can also be inhibited by targeting nucleotide sequences complementary to the regulatory region of the GPCR 4941 (e.g., the GPCR 4941 promoter and/or enhancers) to form triple helical structures that prevent transcription of the GPCR 4941 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the GPCR 4941 protein and interfere with its activity may also be used to modulate or inhibit GPCR 4941 protein function. Such antibodies may be generated using standard techniques described herein, against the GPCR 4941 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the GPCR 4941 protein. Antibodies that are specific for one or more extracellular domains of the GPCR 4941 protein, for example, and that interfere with its activity, are particularly useful in treating cardiovascular or tumorigenic disease. Such antibodies

are especially efficient because they can access the target domains directly from the bloodstream. Any of the administration techniques described below which are appropriate for peptide administration may be utilized to effectively administer inhibitory target gene antibodies to their site of action.

(ii) Methods for Restoring or Enhancing Target Gene Activity

Genes that cause cardiovascular or tumorigenic disease may be underexpressed within cardiovascular or tumorigenic disease situations. Alternatively, the activity of the protein products of such genes may be decreased, leading to the development of cardiovascular or tumorigenic disease symptoms. Such down-regulation of gene expression or decrease of protein activity might have a causative or exacerbating effect on the disease state.

In some cases, genes that are up-regulated in the disease state might be exerting a protective effect. A-variety of techniques may be used to increase the expression, synthesis, or activity of genes and/or proteins that exert a protective effect in response to cardiovascular or tumorigenic disease conditions.

Described in this section are methods whereby the level GPCR 4941 activity may be increased to levels wherein cardiovascular or tumorigenic disease symptoms are ameliorated. The level of GPCR 4941 activity may be increased, for example, by either increasing the level of GPCR 4941 gene expression or by increasing the level of active GPCR 4941 protein which is present.

For example, a GPCR 4941 protein, at a level sufficient to ameliorate cardiovascular or tumorigenic disease symptoms may be administered to a patient exhibiting such symptoms. Any of the techniques discussed below may be used for such administration. One of skill in the art will readily know how to determine the concentration of effective, non-toxic doses of the GPCR 4941 protein, utilizing techniques such as those described below.

Additionally, RNA sequences encoding a GPCR 4941 protein may be directly administered to a patient exhibiting cardiovascular or tumorigenic disease symptoms, at a concentration sufficient to produce a level of GPCR 4941 protein such that cardiovascular or tumorigenic disease symptoms are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a GPCR 4941 gene, or a portion thereof, that directs the production of a normal GPCR 4941 protein with GPCR 4941 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of GPCR 4941 gene sequences into human cells.

Cells, preferably, autologous cells, containing GPCR 4941 expressing gene sequences may then be introduced or reintroduced into the subject at positions which allow for the amelioration of cardiovascular or tumorigenic disease symptoms. Such cell replacement techniques may be preferred, for example, when the gene product is a secreted, extracellular gene product.

C. Pharmacogenomics

The GPCR 4941 molecules of the present invention, as well as agents, or modulators which have a stimulatory or inhibitory effect on GPCR 4941 activity (e.g., GPCR 4941 gene expression) as identified by a screening assay described herein can be administered to individuals to treat (prophylactically or therapeutically) GPCR 4941-associated disorders (e.g., cardiovascular disorder, an endothelial cell disorder, or a tumorigenic disorder) associated with aberrant or unwanted GPCR 4941 activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a GPCR 4941 molecule or a GPCR 4941 modulator as well as tailoring the dosage and/or therapeutic regimen of treatment with a GPCR 4941 molecule or GPCR 4941 modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisnis. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a GPCR 4941 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a GPCR 4941 molecule or GPCR 4941 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a GPCR 4941 molecule or GPCR 4941 modulator, such as a modulator identified by one of the exemplary screening assays described herein.

4. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

A. Chromosome Mapping

Once the sequence (or a portion of the sequence) of a gene has been isolated, this sequence can be used to map the location of the gene on a chromosome. This process is called chromosome mapping. Accordingly, portions or fragments of the GPCR 4941 nucleotide sequences, described herein, can be used to map the location of the GPCR 4941 genes on a chromosome. The mapping of the GPCR 4941 sequences to chromosomes is an important first step in correlating these sequences with genes associated with disease. The GPCR 4941 gene has been mapped to human chromosome position 2q21-q22 (McKee et al. Genomics (1997) 46:426-434).

Briefly, GPCR 4941 genes can be mapped to chromosomes by preparing PCR primers (preferably 15-25 bp in length) from the GPCR 4941 nucleotide sequences. Computer analysis of the GPCR 4941 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the GPCR 4941 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (DEustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GPCR 4941 nucleotide sequences to design oligonucleotide primers, sublocalization can be achieved with panels of fragments from specific chromosomes. Other mapping strategies which can similarly be used to map a GPCR 4941 sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the GPCR 4941 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

B. Tissue Typing

The GPCR 4941 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the GPCR 4941 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The GPCR 4941 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of GPCR 4941 gene sequences can comfortably provide positive individual identification with a panel of perhaps 10 to 1,000 primers which each yield a noncoding amplified sequence of 100 bases. If predicted coding sequences, such as those in SEQ ID NO:1 are used, a more appropriate number of primers for positive individual identification would be 500-2,000.

If a panel of reagents from GPCR 4941 nucleotide sequences described herein is used to generate a unique identification database for an individual, those same reagents can later be used to identify tissue from that individual. Using the unique identification database, positive identification of the individual, living or dead, can be made from extremely small tissue samples.

C: Use of Partial GPCR 4941 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of GPCR 4941 gene sequences are particularly appropriate for this use as greater numbers of polymorphisms occur in the noncoding regions, making it easier to differentiate individuals using this technique. Examples of polynucleotide reagents include the GPCR 4941 nucleotide sequences or portions thereof, e.g., fragments derived from the noncoding regions having a length of at least 20 bases, preferably at least 30 bases.

The GPCR 4941 nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such GPCR 4941 probes can be used to identify tissue by species and/or by organ type.

In a similar fashion, these reagents, e.g., GPCR 4941 primers or probes can be used to screen tissue culture for contamination (i.e. screen for the presence of a mixture of different types of cells in a culture).

5. Recombinant Expression Vectors and Host Cells

The methods of the invention include the use of vectors, preferably expression vectors, containing a nucleic acid encoding a GPCR 4941 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most: commonly used form of vector. However, the methods of the invention may include other forms of expression vectors, such as viral vectors (e.g., replication defective retroviruses, adenoviruses and adeno-associated viruses), which serve equivalent functions.

The recombinant expression vectors used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., GPCR 4941 proteins, mutant forms of GPCR 4941 proteins, fusion proteins, and the like).

The recombinant expression vectors used in the methods of the invention can be designed for expression of GPCR 4941 proteins in prokaryotic or eukaryotic cells, e.g., for use in the cell-based assays of the invention. For example, GPCR 4941 proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX.(Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST); maltose E binding protein, or protein A, respectively, to the target recombinant protein.

Purified fusion proteins can be utilized in GPCR 4941 activity assays, (e.g., direct assays or competitive assays described in detail below), or to generate antibodies specific for GPCR 4941 proteins, for example. In a preferred embodiment, a GPCR 4941 fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the GPCR 4941 expression vector is a yeast expression vector. Examples of vectors for expression in yeast S. cerevisiae include pYepSec 1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, GPCR 4941 proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), endothelial cell-specific promoters (e.g., KDR/flk promoter; U.S. Pat. No. 5,888,765), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The expression characteristics of an endogenous GPCR 4941 gene within a cell line or microorganism may be modified by inserting a heterologous DNA regulatory element into the genome of a stable cell line or cloned microorganism such that the inserted regulatory element is operatively linked with the endogenous GPCR 4941 gene. For example, an endogenous GPCR 4941 gene which is normally “transcriptionally silent”, i.e., a GPCR 4941 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous GPCR 4941 gene may be activated by insertion of a promiscuous regulatory element that works across cell types.

A heterologous regulatory element may be inserted into a stable cell line or cloned microorganism, such that it is operatively linked with an endogenous GPCR 4941 gene, using techniques, such as targeted homologous recombination, which are well known to those of skill in the art, and described, e.g., in Chappel, U.S. Pat. No. 5,272,071; PCT publication No. WO 91/06667, published May 16, 1991.

The methods of the invention use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to GPCR 4941 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect the methods of the invention pertains to the use of host cells into which a GPCR 4941 nucleic acid molecule of the invention is introduced, e.g., a GPCR 4941 nucleic acid molecule within a recombinant expression vector or a GPCR 4941 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a GPCR 4941 protein can be expressed in bacterial cells such as E. coli, insect cells, yeast or mammalian cells (such as human umbilical vein endothelial cells (HUVEC), human microvascular endothelial cells (HMVEC), Chinese hamster ovary cells (CHO), human ovarian surface epithelial (HOSE) cells, or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, puromycin, zeomycin and methotrexate. Nucleic acid encoding a selectable marker can be introduced into a host cell on the same vector as that encoding a GPCR 4941 protein or can be introduced on a separate vector. Cells stably transfected with the introduced nucleic acid can be identified by drug selection (e.g., cells that have incorporated the selectable marker gene will survive, while the other cells die).

A host cell used in the methods of the invention, such as a prokaryotic or eukaryotic host cell in culture, can be used to produce (i.e., express) a GPCR 4941 protein. Accordingly, the invention further provides methods for producing a GPCR 4941 protein using the host cells of the invention. In one embodiment, the method comprises culturing the host cell of the invention (into which a recombinant expression vector encoding a GPCR 4941 protein has been introduced) in a suitable medium such that a GPCR 4941 protein is produced. In another embodiment, the method further comprises isolating a GPCR 4941 protein from the medium or the host cell.

6. Cell- and Animal-Based Model Systems

Described herein are cell- and animal-based systems which act as models for cardiovascular or tumorigenic disease. These systems may be used in a variety of applications. For example, the cell- and animal-based model systems may be used to further characterize differentially expressed genes associated with cardiovascular or tumorigenic disease, e.g., GPCR 4941. In addition, animal- and cell-based assays may be used as part of screening strategies designed to identify compounds which are capable of ameliorating cardiovascular or tumorigenic disease symptoms, as described, below. Thus, the animal- and cell-based models may be used to identify drugs, pharmaceuticals, therapies and interventions which may be effective in treating cardiovascular disease. Furthermore, such animal models may be used to determine the LD50 and the ED50 in animal subjects, and such data can be used to determine the in vivo efficacy of potential cardiovascular or tumorigenic disease treatments.

A. Animal-Based Systems

Animal-based model systems of cardiovascular or tumorigenic disease may include, but are not limited to, non-recombinant and engineered transgenic animals.

Non-recombinant animal models for cardiovascular disease may include, for example, genetic models. Such genetic cardiovascular disease models may include, for example, apoB or apoR deficient pigs (Rapacz, et al., 1986, Science 234:1573-1577) and Watanabe heritable hyperlipidemic (WHHL) rabbits (Kita et al., 1987, Proc. Natl. Acad. Sci USA 84: 5928-5931). Transgenic mouse models in cardiovascular disease and angiogenesis are reviewed in Carmeliet, P. and Collen, D. (2000) J. Pathol. 190:387-405.

Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty. Animal models of cardiovascular disease also include rat myocardial infarction models (described in, for example, Schwarz, ER et al. (2000) J. Am. Coll. Cardiol. 35:1323-1330) and models of chromic cardiac ischemia in rabbits (described in, for example, Operschall, C et al. (2000) J. Appl. Physiol. 88:1438-1445).

Models for studying angiogenesis in vivo include tumor cell-induced angiogenesis and tumor metastasis (Hoffman, R M (1998-99) Cancer Metastasis Rev. 17:271-277; Holash, J et al. (1999) Oncogene 18:5356-5362; Li, C Y et al. (2000) J. Natl Cancer Inst. 92:143-147), matrix induced angiogenesis (U.S. Pat. No. 5,382,514), the disc angiogenesis system (Kowalski, J. et al. (1992) Exp. Mol. Pathol. 56:1-19), the rodent mesenteric-window angiogenesis assay (Norrby, K (1992) EXS 61:282-286), experimental choroidal neovascularization in the rat (Shen, W Y et al. (1998) Br. J. Ophthalmol. 82:1063-1071), and the chick embryo development (Brooks, PC et al. Methods Mol. Biol. (1999) 129:257-269) and chick embryo chorioallantoic membrane (CAM) models (McNatt L G et al. (1999) J. Ocul. Pharmacol. Ther. 15:413-423; Ribatti, D et al. (1996) Int. J. Dev. Biol. 40:1189-1197), and are reviewed in Ribatti, D and Vacca, A (1999) Int. J. Biol. Markers 14:207-213.

Animal based models for studying tumorigenesis in vivo are well known in the art (reviewed in Animal Models of Cancer Predisposition Syndromes, Hiai, H and Hino, 0 (eds.) 1999, Progress in Experimental Tumor Research, Vol. 35; Clarke A R Carcinogenesis (2000) 21:435-41) and include, for example, carcinogen-induced tumors (Rithidech, K et al. Mutat Res (1999) 428:33-39; Miller, M L et al. Environ Mol Mutagen (2000) 35:319-327), injection and/or transplantation of tumor cells into an animal, as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221). Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294).

Additionally, animal models exhibiting cardiovascular or tumorigenic disease symptoms may be engineered by using, for example, GPCR 4941 gene sequences described above, in conjunction with techniques for producing transgenic animals that are well known to those of skill in the art. For example, GPCR 4941 gene sequences may be introduced into, and overexpressed in, the genome of the animal of interest, or, if endogenous GPCR 4941 gene sequences are present, they may either be overexpressed or, alternatively, be disrupted in order to underexpress or inactivate GPCR 4941 gene expression, such as described for the disruption of apoE in mice (Plump et al., 1992, Cell 71: 343-353).

The host cells of the invention can also be used to produce non-human transgenic animals. For example, in one embodiment, a host cell of the invention is a fertilized oocyte or an embryonic stem cell into which GPCR 4941-coding sequences have been introduced. Such host cells can then be used to create non-human transgenic animals in which exogenous GPCR 4941 sequences have been introduced into their genome or homologous recombinant animals in which endogenous GPCR 4941 sequences have been altered. Such animals are useful for studying the function and/or activity of a GPCR 4941 and for identifying and/or evaluating modulators of GPCR 4941 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous GPCR 4941 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a GPCR 4941-encoding nucleic acid into the male pronuclei of a fertilized oocyte, e.g., by microinjection, retroviral infection, and allowing the oocyte to develop in a pseudopregnant female foster animal. The GPCR 4941 cDNA sequence of SEQ ID NO:1 or 3 can be introduced as a transgene into the genome of a non-human animal. Alternatively, a nonhuman homologue of a human GPCR 4941 gene, such as a mouse or rat GPCR 4941 gene, can be used as a transgene. Alternatively, a GPCR 4941 gene homologue, such as another GPCR 4941 family member, can be isolated based on hybridization to the GPCR 4941 cDNA sequences of SEQ ID NO:1 or 3 and used as a transgene. Intronic sequences and polyadenylation signals can also be included in the transgene to increase the efficiency of expression of the transgene. A tissue-specific regulatory sequence(s) can be operably linked to a GPCR 4941 transgene to direct expression of a GPCR 4941 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a GPCR 4941 transgene in its genome and/or expression of GPCR 4941 mRNA in tissues or cells of the animals. A transgenic founder animal can then be used to breed additional animals carrying the transgene. Moreover, transgenic animals carrying a transgene encoding a GPCR 4941 protein can further be bred to other transgenic animals carrying other transgenes.

To create a homologous recombinant animal, a vector is prepared which contains at least a portion of a GPCR 4941 gene into which a deletion, addition or substitution has been introduced to thereby alter, e.g., functionally disrupt, the GPCR 4941 gene. The GPCR 4941 gene can be a human gene (e.g., the cDNA of SEQ ID NO:1 or 3), but more preferably, is a non-human homologue of a human GPCR 4941 gene (e.g., a cDNA isolated by stringent hybridization with the nucleotide sequence of SEQ ID NO:1 or 3). For example, a mouse GPCR 4941 gene can be used to construct a homologous recombination nucleic acid molecule, e.g., a vector, suitable for altering an endogenous GPCR 4941 gene in the mouse genome. In a preferred embodiment, the homologous recombination nucleic acid molecule is designed such that, upon homologous recombination, the endogenous GPCR 4941 gene is functionally disrupted (i.e., no longer encodes a functional protein; also referred to as a “knock out” vector). Alternatively, the homologous recombination nucleic acid molecule can be designed such that, upon homologous recombination, the endogenous GPCR 4941 gene is mutated or otherwise altered but still encodes functional protein (e.g., the upstream regulatory region can be altered to thereby alter the expression of the endogenous GPCR 4941 protein). In the homologous recombination nucleic acid molecule, the altered portion of the GPCR 4941 gene is flanked at its 5′ and 3′ ends by additional nucleic acid sequence of the GPCR 4941 gene to allow for homologous recombination to occur between the exogenous GPCR 4941 gene carried by the homologous recombination nucleic acid molecule and an endogenous GPCR 4941 gene in a cell, e.g., an embryonic stem cell. The additional flanking GPCR 4941 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (i987) Cell 51:503 for a description of homologous recombination vectors). The homologous recombination nucleic acid molecule is introduced into a cell, e.g., an embryonic stem cell line (e.g., by electroporation) and cells in which the introduced GPCR 4941 gene has homologously recombined with the endogenous GPCR 4941 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced, which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The GPCR 4941 transgenic animals that express GPCR 4941 mRNA or a GPCR 4941 peptide (detected immunocytochemically, using antibodies directed against GPCR 4941 epitopes) at easily detectable levels should then be further evaluated to identify those animals which display characteristic cardiovascular or tumorigenic disease symptoms. Such cardiovascular disease symptoms may include, for example, increased prevalence and size of fatty streaks and/or cardiovascular disease plaques. Tumorigenic disease symptoms include, for example, tumor burden, invasion and/or metastasis.

Additionally, specific cell types (e.g., endothelial cells, monocytes, ovarian cells) within the transgenic animals may be analyzed and assayed for cellular phenotypes characteristic of cardiovascular or tumorigenic disease. In the case of endothelial cells, such phenotypes include, but are not limited to cell proliferation, migration, angiogenesis, production of proinflammatory growth factors and cytokines, and adhesion to inflammatory cells. In the case of monocytes, such phenotypes may include but are not limited to increases in rates of LDL uptake, adhesion to endothelial cells, transmigration, foam cell formation, fatty streak formation, and production of foam cell specific products. Cellular phenotypes associated with a tumorigenic disorder include, for example, dysregulated proliferation and migration, anchorage independent growth, and loss of contact inhibition. Cellular phenotypes may include a particular cell type's pattern of expression of genes associated with cardiovascular disease as compared to known expression profiles of the particular cell type in animals exhibiting cardiovascular or tumorigenic disease symptoms.

B. Cell-Based Systems

Cells that contain and express GPCR 4941 gene sequences which encode a GPCR 4941 protein, and, further, exhibit cellular phenotypes associated with cardiovascular or tumorigenic disease, may be used to identify compounds that exhibit anti-cardiovascular disease or anti-tumorigenic disease activity. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); ovarian cells such as CHO cells, e.g., CHO-Ki (ATCC# CCL-61), NOV-31 (ATCC# CRL-11733), ES-2 (ATCC# CRL-1978), MDAH 2774 (ATCC# CRL-10303), TOV-112D (ATCC# CRL-11731), Caov-3 (ATCC# HTB-75), SK-OV-3 (ATCC# HTB-77), NIH:OVCAR-3 (ATCC# HTB-161), human ovarian surface epithelial (HOSE) cells, and A2780 cells; tumor cell lines such as HT-1080 (ATCC# CCL-121), HCT-15 (ATCC# CCL-225), HCC70 (ATCC# CRL-2315), M059J (ATCC# CRL-2366), and NC1-N417 (ATCC# CRL-5809); as well as generic mammalian cell lines such as HeLa cells and COS cells, e.g., COS-7 (ATCC# CRL-1651). Further, such cells may include recombinant, transgenic cell lines. For example, the cardiovascular or tumorigenic disease animal models of the invention, discussed above, may be used to generate cell lines, containing one or more cell types involved in cardiovascular or tumorigenic disease, that can be used as cell culture models for this disorder. While primary cultures derived from the cardiovascular or tumorigenic disease transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in cardiovascular or tumorigenic disease may be transfected with sequences capable of increasing or decreasing the amount of GPCR 4941 gene expression within the cell. For example, GPCR 4941 gene sequences may be introduced into, and overexpressed in, the genome of the cell of interest, or, if endogenous GPCR 4941 gene sequences are present, they may be either overexpressed or, alternatively disrupted in order to underexpress or inactivate GPCR 4941 gene expression.

In order to overexpress a GPCR 4941 gene, the coding portion of the GPCR 4941 gene may be ligated to a regulatory sequence which is capable of driving gene expression in the cell type of interest, e.g., an endothelial cell or an ovarian cell. Such regulatory regions will be well known to those of skill in the art, and may be utilized in the absence of undue experimentation. Recombinant methods for expressing target genes are described above.

For underexpression of an endogenous GPCR 4941 gene sequence, such a sequence may be isolated and engineered such that when reintroduced into the genome of the cell type of interest, the endogenous GPCR 4941 alleles will be inactivated. Preferably, the engineered GPCR 4941 sequence is introduced via gene targeting such that the endogenous GPCR 4941 sequence is disrupted upon integration of the engineered GPCR 4941 sequence into the cell's genome. Transfection of host cells with GPCR 4941 genes is discussed, above.

Cells treated with compounds or transfected with GPCR 4941 genes can be examined for phenotypes associated with cardiovascular or tumorigenic disease. In the case of monocytes, such phenotypes include but are not limited to increases in rates of LDL uptake, adhesion to endothelial cells, transmigration, foam cell formation, fatty streak formation, and production by foam cells of growth factors such as bFGF, IGF-I, VEGF, IL-1, M-CSF, TGFβ, TGFα, TNFα, HB-EGF, PDGF, IFN-γ, and GM-CSF. Transmigration rates, for example, may be measured using the in vitro system of Navab et al. (1988) J. Clin. Invest. 82:1853-1863, by quantifying the number of monocytes that migrate across the endothelial monolayer and into the collagen layer of the subendothelial space.

Similarly, endothelial cells can be treated with test compounds or transfected with genetically engineered GPCR 4941 genes. The endothelial cells can then be examined for phenotypes associated with cardiovascular disease, including, but not limited to changes in cellular morphology, cell proliferation, cell migration, and mononuclear cell adhesion; or for the effects on production of other proteins involved in cardiovascular disease such as adhesion molecules (e.g., ICAM, VCAM, E-selectin), growth factors and cytokines (e.g., PDGF, IL-β, TNFα, MCF), and proteins involved in angiogenesis (e.g., FLK, FLT).

Moreover, cells (e.g., ovarian cells) can be treated with test compounds or transfected with genetically engineered GPCR 4941 genes and examined for phenotypes associated with tumorigenic disease, including, but not limited to changes in cellular morphology, cell proliferation, cell migration, cell transformation, anchorage independent growth, and loss of contact inhibition.

Transfection of GPCR 4941 nucleic acid may be accomplished by using standard techniques (described in, for example, Ausubel (1989) supra). Transfected cells should be evaluated for the presence of the recombinant GPCR 4941 gene sequences, for expression and accumulation of GPCR 4941 mRNA, and for the presence of recombinant GPCR 4941 protein production. In instances wherein a decrease in GPCR 4941 gene expression is desired, standard techniques may be used to demonstrate whether a decrease in endogenous GPCR 4941 gene expression and/or in GPCR 4941 protein production is achieved.

Cellular models for the study of cardiovascular disease and angiogenesis include models of endothelial cell differentiation on Matrigel (Baatout, S. et al. (1996) Rom. J. Intern. Med. 34:263-269; Benelli, R et al. (1999) Int. J. Biol. Markers 14:243-246), embryonic stem cell models of vascular morphogenesis (Doetschman, T. et al. (1993) Hypertension 22:618-629), the culture of microvessel fragments in physiological gels (Hoying, J B et al. (1996) In Vitro Cell Dev. Biol. Anim. 32: 409-419; U.S. Pat. No. 5,976,782), and the treatment of endothelial cells and smooth muscle cells with atherogenic and angiogenic factors including growth factors and cytokines (e.g., IL-1β, PDGF, TNFα, VEGF), homocysteine, and LDL. In vitro angiogenesis models are described in, for example, Black, A F et al. (1999) Cell Biol. Toxicol. 15:81-90.

Cellular models for the study of tumorigenesis are known in the art, and include cell lines derived from clinical tumors, cells exposed to chemotherapeutic agents, cells exposed to carcinogenic agents, and cell lines with genetic alterations in growth regulatory genes, for example, oncogenes (e.g., ras) and tumor suppressor genes (e.g., p53).

7. Pharmaceutical Compositions

Active compounds for use in the methods of the invention can be incorporated into pharmaceutical compositions suitable for administration. As used herein, the language “active compounds” includes GPCR 4941 nucleic acid molecules, fragments of GPCR 4941 proteins, and anti-GPCR 4941 antibodies, as well as identified compounds that modulate GPCR 4941 gene expression, synthesis, and/or activity. Such compositions typically comprise the compound, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a GPCR 4941 protein or a GPCR 4941 ligand) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. In one embodiment, a therapeutically effective dose refers to that amount of an active compound sufficient to result in amelioration of symptoms of cardiovascular disease.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams pet mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator of GPCR 4941 activity is administered in combination with other agents (e.g., a small molecule), or in conjunction with another, complementary treatment regime. For example, in one embodiment, a modulator of GPCR 4941 activity is used to treat a tumorigenic disorder, e.g., ovarian cancer. Accordingly, modulation of GPCR 4941 activity may be used in conjunction with, for example, chemotherapeutic agents, radiation treatment, and/or anti-angiogenesis agents. In another embodiment, a modulator of GPCR 4941 activity is used to treat a cardiovascular disorder, and may be used in conjunction with, for example, vasoactive agents, e.g., modulators of coagulation and/or vessel radius.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (CDDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

8. Isolated Nucleic Acid Molecules

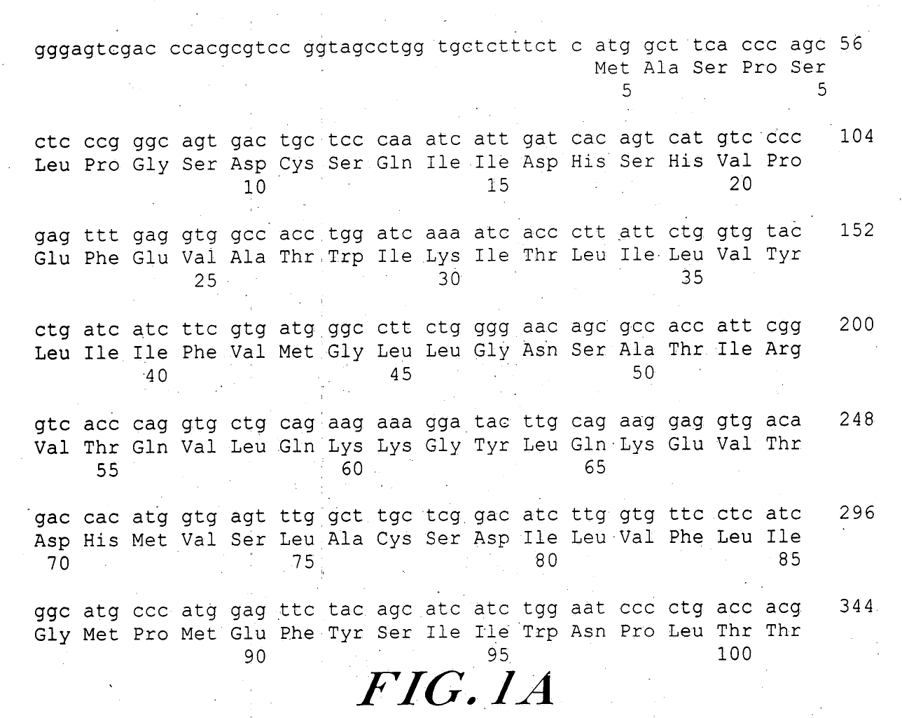

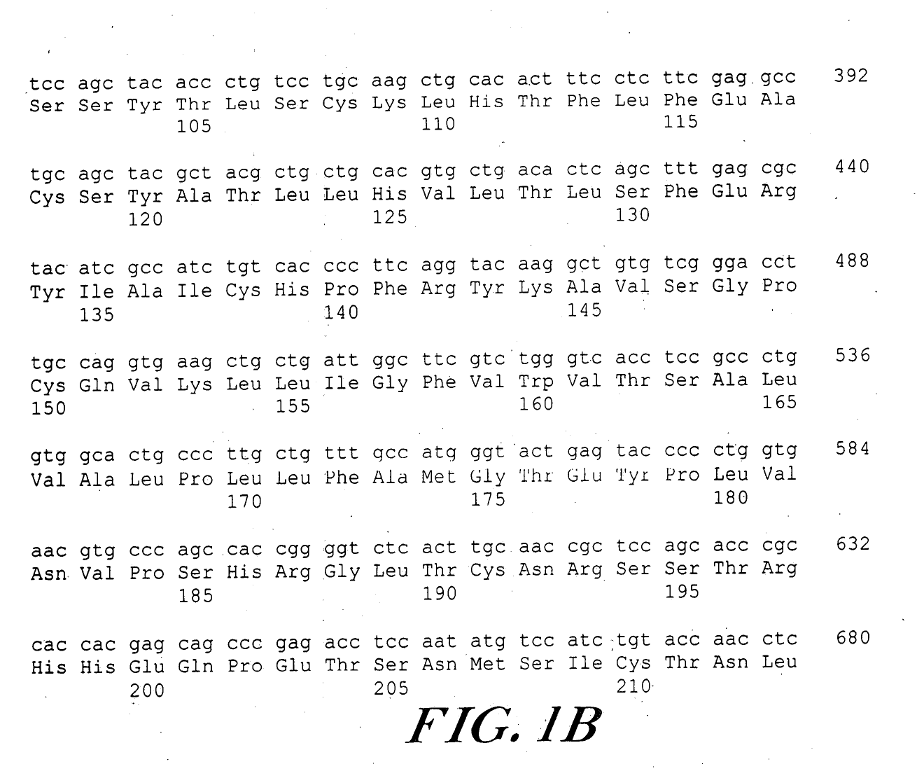

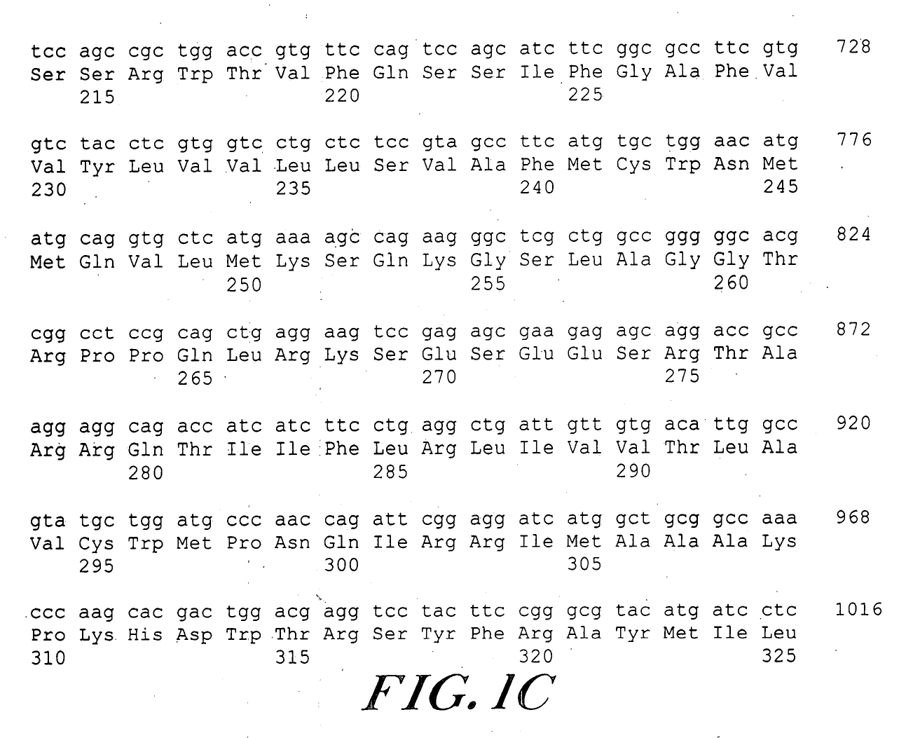

The nucleotide sequence of the isolated human GPCR 4941 cDNA and the predicted amino acid sequence of the human GPCR 4941 polypeptide are shown in FIG. 1 and in SEQ ID NOs:3 and 2, respectively. The nucleotide sequence encoding human GPCR 4941 is identical to GPR39, GenBank Accession Number AF034633 (McKee et al. Genomics (1997) 46:426-434).

The human GPCR 4941 gene, which is approximately 2528 nucleotides in length, encodes a protein having a molecular weight of approximately 50 kD and which is approximately 453 amino acid residues in length.

The methods of the invention include the use of isolated nucleic acid molecules that encode GPCR 4941 proteins or biologically active portions thereof, as well as nucleic acid fragments sufficient for use as hybridization probes to identify GPCR 4941-encoding nucleic acid molecules (e.g., GPCR 4941 mRNA) and fragments for use as PCR primers for the amplification or mutation of GPCR 4941 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated GPCR 4941 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule used in the methods of the present invention, e.g., a nucleic acid molecule having the nucleotide sequence of SEQ ID NO:1 or 3, or a portion thereof, can be isolated using standard molecular biology techniques and the sequence information provided herein. Using all or portion of the nucleic acid sequence of SEQ ID NO:1 or 3, as a hybridization probe, GPCR 4941 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 or 3 can be isolated by the polymerase chain reaction (PCR) using synthetic oligonucleotide primers designed based upon the sequence of SEQ ID NO:1 or 3.

A nucleic acid used in the methods of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GPCR 4941 nucleotide sequences can be prepared by standard synthetic techniques, e.g., using an automated DNA synthesizer.

In a preferred embodiment, an isolated nucleic acid molecule used in the methods of the invention comprises the nucleotide sequence shown in SEQ ID NO:1 or 3. This cDNA may comprise sequences encoding the human SPRP-1 protein (i.e., “the coding region”, from nucleotides 42-1403), as well as 5′ untranslated sequences (nucleotides 1-41) and 3′ untranslated sequences (nucleotides 1404-2528) of SEQ ID NO:3. Alternatively, the nucleic acid molecule can comprise only the coding region of SEQ ID NO:3 (e.g., nucleotides 42-1403, corresponding to SEQ ID NO:1).

In another preferred embodiment, an isolated nucleic acid molecule used in the methods of the invention comprises a nucleic acid molecule which is a complement of the nucleotide sequence shown in SEQ ID NO:1 or 3, or a portion of any of this nucleotide sequence. A nucleic acid molecule which is complementary to the nucleotide sequence shown in SEQ ID NO:1 or 3 is one which is sufficiently complementary to the nucleotide sequence shown in SEQ ID NO:1 or 3 such that it can hybridize to the nucleotide sequence shown in SEQ ID NO:1 or 3, thereby forming a stable duplex.

In still another preferred embodiment, an isolated nucleic acid molecule used in the methods of the present invention comprises a nucleotide sequence which is at least about 30%, 35%, 40%, 45%, 50%, 55%, 60%, 65%, 70%, 75%, 80%, 85%, 90%, 95%, 98% or more identical to the entire length of the nucleotide sequence shown in SEQ ID NO:1 or 3, or a portion of any of this nucleotide sequence.

Moreover, a nucleic acid molecule used in the methods of the invention can comprise only a portion of the nucleic acid sequence of SEQ ID NO:1 or 3, for example, a fragment which can be used as a probe or primer or a fragment encoding a portion of a GPCR 4941 protein, e.g., a biologically active portion of a GPCR 4941 protein. The nucleotide sequence determined from the cloning of the GPCR 4941 gene allows for the generation of probes and primers designed for use in identifying and/or cloning other GPCR 4941 family members, as well as GPCR 4941 homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 or 3, of an anti-sense sequence of SEQ ID NO:1 or 3, or of a naturally occurring allelic variant or mutant of SEQ ID NO:1 or 3. In one embodiment, a nucleic acid molecule of the present invention comprises a nucleotide sequence which is greater than 100, 100-200, 200-300, 300-400, 400-500, 500-600, 600-700, 700-800, 800-900, 900-1000, 1000-1100, 1100-120, 1200-1300, 1300 or more nucleotides in length and hybridizes under stringent hybridization conditions to a nucleic acid molecule of SEQ ID NO:1 or 3.

Probes based on the GPCR 4941 nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a GPCR 4941 protein, such as by measuring a level of a GPCR 4941-encoding nucleic acid in a sample of cells from a subject e.g., detecting GPCR 4941 mRNA levels or determining whether a genomic GPCR 4941 gene has been mutated or deleted.

A nucleic acid fragment encoding a “biologically active portion of a GPCR 4941 protein” can be prepared by isolating a portion of the nucleotide sequence of SEQ ID NO:1 or 3 which encodes a polypeptide having a GPCR 4941 biological activity (the biological activities of the GPCR 4941 protein is described herein), expressing the encoded portion of the GPCR 4941 protein (e.g., by recombinant expression in vitro) and assessing the activity of the encoded portion of the GPCR 4941 protein.

The methods of the invention further encompass the use of nucleic acid molecules that differ from the nucleotide sequence shown in SEQ ID NO:1 or 3, due to degeneracy of the genetic code and thus encode the same GPCR 4941 protein as those encoded by the nucleotide sequence shown in SEQ ID NO:1 or 3. In another embodiment, an isolated nucleic acid molecule of the invention has a nucleotide sequence encoding a protein having an amino acid sequence shown in SEQ ID NO:2.

In addition to the GPCR 4941-nucleotide sequence shown in SEQ ID NO:1 or 3, it will be appreciated by those skilled in the art that DNA sequence polymorphisms that lead to changes in the amino acid sequences of the GPCR 4941 protein may exist within a population (e.g., the human population). Such genetic polymorphism in the GPCR 4941 gene may exist among individuals within a population due to natural allelic variation. As used herein, the terms “gene” and “recombinant gene” refer to nucleic acid molecules which include an open reading frame encoding a GPCR 4941 protein, preferably a mammalian GPCR 4941 protein, and can further include non-coding regulatory sequences, and introns.

Allelic variants of human GPCR 4941 include both functional and non-functional GPCR 4941 proteins. Functional allelic variants are naturally occurring amino acid sequence variants of the human GPCR 4941 protein that maintain the ability to bind a GPCR 4941 ligand or substrate and/or modulate cell proliferation and/or migration mechanisms. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human GPCR 4941 protein that do not have the ability to either bind a GPCR 4941 ligand or substrate and/or modulate cell proliferation and/or migration mechanisms. Non-functional allelic variants will typically contain a non-conservative substitution, a deletion, or insertion or premature truncation of the amino acid sequence of SEQ ID NO:2, or a substitution, insertion or deletion in critical residues or critical regions.

The methods of the present invention may further use non-human orthologues of the human GPCR 4941 protein. Orthologues of the human GPCR 4941 protein are proteins that are isolated from non-human organisms and possess the same GPCR 4941 ligand binding and/or modulation of cell proliferation and/or migration mechanisms of the human GPCR 4941 protein. Orthologues of the human GPCR 4941 protein can readily be identified as comprising an amino acid sequence that is substantially identical to SEQ ID NO:2.

Moreover, nucleic acid molecules encoding other GPCR 4941 family members and, thus, which have a nucleotide sequence which differs from the GPCR 4941 sequence of SEQ ID NO:1 or 3 are intended to be within the scope of the invention. For example, another GPCR 4941 cDNA can be identified based on the nucleotide sequence of human GPCR 4941. Moreover, nucleic acid molecules encoding GPCR 4941 proteins from different species, and which, thus, have a nucleotide sequence which differs from the GPCR 4941 sequence of SEQ ID NO:1 or 3 are intended to be within the scope of the invention. For example, a mouse GPCR 4941 cDNA can be identified based on the nucleotide sequence of human GPCR 4941.

Nucleic acid molecules corresponding to natural allelic variants and homologues of the GPCR 4941 cDNA of the invention can be isolated based on their homology to the GPCR 4941 nucleic acid disclosed herein using the cDNAs disclosed herein, or a portion thereof, as a hybridization probe according to standard hybridization techniques under stringent hybridization conditions. Nucleic acid molecules corresponding to natural allelic variants and homologues of the GPCR 4941 cDNA of the invention can further be isolated by mapping to the same chromosome or locus as the GPCR 4941 gene.

Accordingly, in another embodiment, an isolated nucleic acid molecule used in the methods of the invention is at least 15, 20, 25, 30 or more nucleotides in length and hybridizes under stringent conditions to the nucleic acid molecule comprising the nucleotide sequence of SEQ ID NO:1 or 3. In other embodiment, the nucleic acid is at least 30, 50, 100, 150, 200, 250, 300, 350, 400, 450, 500, 550, 600, 650, 700, 750, 800, 1000, 1200, or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see, e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:1 or 3 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the GPCR 4941 sequences that may exist in the population, the skilled artisan will further appreciate that changes can be introduced by mutation into the nucleotide sequence of SEQ ID NO:1 or 3, thereby leading to changes in the amino acid sequence of the encoded GPCR 4941 protein, without altering the functional ability of the GPCR 4941 protein. For example, nucleotide substitutions leading to amino acid substitutions at “non-essential” amino acid residues can be made in the sequence of SEQ ID NO:1 or 3. A “non-essential” amino acid residue is a residue that can be altered from the wild-type sequence of GPCR 4941 (e.g., the sequence of SEQ ID NO:2) without altering the biological activity, whereas an “essential” amino acid residue is required for biological activity. For example, amino acid residues that are conserved among the GPCR 4941 proteins of the present invention are predicted to be particularly unamenable to alteration. Furthermore, additional amino acid residues that are conserved between the GPCR 4941 proteins of the present invention and other members of the G protein-coupled receptor family (e.g., the growth hormone secretagogue receptor (GHS-R) and the neurotensin receptor (NT-R)) are not likely to be amenable to alteration.

Accordingly, the methods of the invention may include the use of nucleic acid molecules encoding GPCR 4941 proteins that contain changes in amino acid residues that are not essential for activity. Such GPCR 4941 proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 30%, 35%, 40%, 45%, 50%, 55%, 60%, 65%, 70%, 75%, 80%, 85%, 90%, 95%, 98% or more identical to SEQ ID NO:2.

An isolated nucleic acid molecule encoding a GPCR 4941 protein identical to the protein of SEQ ID NO:2 can be created by introducing one or more nucleotide substitutions, additions or deletions into the nucleotide sequence of SEQ ID NO:1 or 3 such that one or more amino acid substitutions, additions or deletions are introduced into the encoded protein. Mutations can be introduced into SEQ ID NO:1 or 3 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a GPCR 4941 protein is preferably replaced with another amino acid residue from the same side chain family. Alternatively, in another embodiment, mutations can be introduced randomly along all or part of a GPCR 4941 coding sequence, such as by saturation mutagenesis, and the resultant mutants can be screened for GPCR 4941 biological activity to identify mutants that retain activity. Following mutagenesis of SEQ ID NO:1 or 3, the encoded protein can be expressed recombinantly and the activity of the protein can be determined.

In a preferred embodiment, a mutant GPCR 4941 protein can be assayed for the ability to (1) interact with a non-GPCR 4941 protein molecule, e.g., a GPCR 4941 ligand or substrate; (2) activate a GPCR 4941-dependent signal transduction pathway; or (3) modulate cell proliferation and/or migration mechanisms, or modulate the expression of cell surface adhesion molecules. In addition to the nucleic acid molecules encoding GPCR 4941 proteins described herein, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The-antisense nucleic acid can be complementary to an entire GPCR 4941 coding strand, or to only a portion thereof. In one embodiment, an antisense nucleic acid molecule is antisense to a “coding region” of the coding strand of a nucleotide sequence encoding GPCR 4941. The term “coding region” refers to the region of the nucleotide sequence comprising codons which are translated into amino acid residues (e.g., the coding region of human GPCR 4941 corresponds to SEQ ID NO:1). In another embodiment, the antisense nucleic acid molecule is antisense to a “noncoding region” of the coding strand of a nucleotide sequence encoding GPCR 4941. The term “noncoding region” refers to 5′ and 3′ sequences which flank the coding region that are not translated into amino acids (i.e., also referred to as 5′ and 3′ untranslated regions).

Given the coding strand sequences encoding GPCR 4941 disclosed herein (e.g., SEQ ID NO:1), antisense nucleic acids of the invention can be designed according to the rules of Watson and Crick base pairing. The antisense nucleic acid molecule can be complementary to the entire coding region of GPCR 4941 mRNA, but more preferably is an oligonucleotide which is antisense to only a portion of the coding or noncoding region of GPCR 4941 mRNA. For example, the antisense oligonucleotide can be complementary to the region surrounding the translation start site of GPCR 4941 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

In yet another embodiment, the GPCR 4941 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of GPCR 4941 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of GPCR 4941 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of GPCR 4941 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of GPCR 4941 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

9. Isolated GPCR 4941 Proteins and Anti-GPCR 4941 Antibodies

The methods of the invention include the use of isolated GPCR 4941 proteins, and biologically active portions thereof, as well as polypeptide fragments suitable for use as immunogens to raise anti-GPCR 4941 antibodies.

Isolated proteins used in the methods of the present invention, preferably GPCR 4941 proteins, have an amino acid sequence sufficiently identical to the amino acid sequence of SEQ ID NO:2, or are encoded by a nucleotide sequence sufficiently identical to SEQ ID NO:1 or 3. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, a “GPCR 4941 activity”, “biological activity of GPCR 4941” or “functional activity of GPCR 4941”, refers to an activity exerted by a GPCR 4941 protein, polypeptide or nucleic acid molecule on a GPCR 4941 responsive cell (e.g., an endothelial cell) or tissue, or on a GPCR 4941 protein substrate, as determined in vivo, or in vitro, according to standard techniques. In one embodiment, a GPCR 4941 activity is a direct activity, such as an association with a GPCR 4941 target molecule. As used herein, a “target molecule” or “binding partner” is a molecule with which a GPCR 4941 protein binds or interacts in nature, such that GPCR 4941-mediated function is achieved. A GPCR 4941 target molecule can be a non-GPCR 4941 molecule or a GPCR 4941 protein or polypeptide of the present invention. In an exemplary embodiment, a GPCR 4941 target molecule is a GPCR 4941 ligand. Alternatively, a GPCR 4941 activity is an indirect activity, such as a cellular signaling activity mediated by interaction of the GPCR 4941 protein with a GPCR 4941 ligand. Preferably, a GPCR 4941 activity is the ability to act as a signal transduction molecule and to modulate endothelial cell growth, proliferation, differentiation, migration, and/or tube formation. In another embodiment, a GPCR 4941 activity is the ability to modulate angiogenesis, vascularization and/or tumorigenesis. Accordingly, another embodiment of the invention features isolated GPCR 4941 proteins and polypeptides having a GPCR 4941 activity.

In one embodiment, native GPCR 4941 proteins can be isolated from cells or tissue sources by an appropriate purification scheme using standard protein purification techniques. In another embodiment, GPCR 4941 proteins are produced by recombinant DNA techniques. Alternative to recombinant expression, a GPCR 4941 protein or polypeptide can be synthesized chemically using standard peptide synthesis techniques.

An “isolated” or “purified” protein or biologically active portion thereof is substantially free of cellular material or other contaminating proteins from the cell or tissue source from which the GPCR 4941 protein is derived, or substantially free from chemical precursors or other chemicals when chemically synthesized. The language “substantially free of cellular material” includes preparations of GPCR 4941 protein in which the protein is separated from cellular components of the cells from which it is isolated or recombinantly produced. In one embodiment, the language “substantially free of cellular material” includes preparations of GPCR 4941 protein having less than about 30% (by dry weight) of non-GPCR 4941 protein (also referred to herein as a “contaminating protein”), more preferably less than about 20% of non-GPCR 4941 protein, still more preferably less than about 10% of non-GPCR 4941 protein, and most preferably less than about 5% non-GPCR 4941 protein. When the GPCR 4941 protein or biologically active portion thereof is recombinantly produced, it is also preferably substantially free of culture medium, i.e., culture medium represents less' than about 20%, more preferably less than about 10%, and most preferably less than about 5% of the volume of the protein preparation.

The language “substantially free of chemical precursors or other chemicals” includes preparations of GPCR 4941 protein in which the protein is separated from chemical precursors or other chemicals which are involved in the synthesis of the protein. In one embodiment, the language “substantially free of chemical precursors or other chemicals” includes preparations of GPCR 4941 protein having less than about 30% (by dry weight) of chemical precursors or non-GPCR 4941 chemicals, more preferably less than about 20% chemical precursors or non-GPCR 4941 chemicals, still more preferably less than about 10% chemical precursors or non-GPCR 4941 chemicals, and most preferably less than about 5% chemical precursors or non-GPCR 4941 chemicals.

As used herein, a “biologically active portion” of a GPCR 4941 protein includes a fragment of a GPCR 4941 protein which participates in an interaction between a GPCR 4941 molecule and a non-GPCR 4941 molecule. Biologically active portions of a GPCR 4941 protein include peptides comprising amino acid sequences sufficiently identical to or derived from the amino acid sequence of the GPCR 4941 protein, e.g., the amino acid sequence shown in SEQ ID NO:2, which include less amino acids than the full length GPCR 4941 protein, and exhibit at least one activity of a GPCR 4941 protein. Typically, biologically active portions comprise a domain or motif with at least one activity of the GPCR 4941 protein, e.g., modulating cell signaling mechanisms and/or cell proliferation, differentiation and migration mechanisms. A biologically active portion of a GPCR 4941 protein can be a polypeptide which is, for example, 10, 25, 50, 100, 200, or more amino acids in length. Biologically active portions of a GPCR 4941 protein can be used as targets for developing agents which modulate a GPCR 4941 mediated activity, e.g., a cell signaling mechanism and/or a cell proliferation, differentiation and migration mechanism. A biologically active portion of a GPCR 4941 protein comprises a protein in which regions of the protein are deleted, can be prepared by recombinant techniques and evaluated for one or more of the functional activities of a native GPCR 4941 protein.

In a preferred embodiment, the GPCR 4941 protein has an amino acid sequence shown in SEQ ID NO:2. In other embodiments, the GPCR 4941 protein is substantially identical to SEQ ID NO:2, and retains the functional activity of the protein of SEQ ID NO:2, yet differs in amino acid sequence due to natural allelic variation or mutagenesis, as described in detail in subsection I above. Accordingly, in another embodiment, the GPCR 4941 protein is a protein which comprises an amino acid sequence at least about 30%, 35%, 40%, 45%, 50%, 55%, 60%, 65%, 70%, 75%, 80%, 85%, 90%, 95%, 98% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. e.g., when aligning a second sequence to the GPCR 4941 amino acid sequence of SEQ ID NO:2 having 453 amino acid residues, at least 136, preferably at least 181, more preferably at least 227, even more preferably at least 272, and even more preferably at least 317, 362 or 408 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4:11-17-(1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to GPCR 4941 nucleic acid molecules of the invention. BLAST protein searches can be performed with the XBLAST program, score=100, wordlength=3 to obtain amino acid sequences homologous to GPCR 4941 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The methods of the invention may also use GPCR 4941 chimeric or fusion proteins. As used herein, a GPCR 4941 “chimeric protein” or “fusion protein” comprises a GPCR 4941 polypeptide operatively linked to a non-GPCR 4941 polypeptide. A “GPCR 4941 polypeptide” refers to a polypeptide having an amino acid sequence corresponding to GPCR 4941, whereas a “non-GPCR 4941 polypeptide” refers to a polypeptide having an amino acid sequence corresponding to a protein which is not substantially homologous to the GPCR 4941 protein, e.g., a protein which is different from the GPCR 4941 protein and which is derived from the same or a different organism. Within a GPCR 4941 fusion protein the GPCR 4941 polypeptide can correspond to all or a portion of a GPCR 4941 protein. In a preferred embodiment, a GPCR 4941 fusion protein comprises at least one biologically active portion of a GPCR 4941 protein. In another preferred embodiment, a GPCR 4941 fusion protein comprises at least two biologically active portions of a GPCR 4941 protein. Within the fusion protein, the term “operatively linked” is intended to indicate that the GPCR 4941 polypeptide and the non-GPCR 4941 polypeptide are fused in-frame to each other. The non-GPCR 4941 polypeptide can be fused to the N-terminus or C-terminus of the GPCR 4941 polypeptide.

For example, in one embodiment, the fusion protein is a GST-GPCR 4941 fusion protein in which the GPCR 4941 sequences are fused to the C-terminus of the GST sequences. Such fusion proteins can facilitate the purification of recombinant GPCR 4941.

In another embodiment, the fusion protein is a GPCR 4941 protein containing a heterologous signal sequence at its N-terminus. In certain host cells (e.g., mammalian host cells), expression and/or secretion of GPCR 4941 can be increased through use of a heterologous signal sequence.

The GPCR 4941 fusion proteins used in the methods of the invention can be incorporated into pharmaceutical compositions and administered to a subject in vivo. The GPCR 4941 fusion proteins can be used to affect the bioavailability of a GPCR 4941 ligand. Use of GPCR 4941 fusion proteins may be useful therapeutically for the treatment of disorders caused by, for example, (i) aberrant modification or mutation of a gene encoding a GPCR 4941 protein; (ii) mis-regulation of the GPCR 4941 gene; and (iii) aberrant post-translational modification of a GPCR 4941 protein. In one embodiment, a GPCR 4941 fusion protein may be used to treat a cardiovascular disorder. In another embodiment, a GPCR 4941 fusion protein may be used to treat an endothelial cell disorder, e.g., a disorder associated with aberrant endothelial cell proliferation, differentiation, migration, angiogenesis and/or vascularization. In a further embodiment, a GPCR 4941 fusion protein may be used to treat a tumorigenic disorder, e.g., ovarian cancer.

Moreover, the GPCR 4941-fusion proteins of the invention can be used as immunogens to produce anti-GPCR 4941 antibodies in a subject, to purify GPCR 4941 ligands and in screening assays to identify molecules which inhibit the interaction of GPCR 4941 with a GPCR 4941 substrate.

Preferably, a GPCR 4941 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A GPCR 4941-encoding nucleic acid can be cloned into such an expression vector such that the fusion moiety is linked in-frame to the GPCR 4941 protein.

The methods of the present invention may also include the use of variants of the GPCR 4941 protein which function as either GPCR 4941 agonists (mimetics) or as GPCR 4941 antagonists. Variants of the GPCR 4941 protein can be generated by mutagenesis, e.g., discrete point mutation or truncation of a GPCR 4941 protein. An agonist of the GPCR 4941 protein can retain substantially the same, or a subset, of the biological activities of the naturally occurring form of a GPCR 4941 protein. An antagonist of a GPCR 4941 protein can inhibit one or more of the activities of the naturally occurring form of the GPCR 4941 protein by, for example, competitively modulating a GPCR 4941-mediated activity of a GPCR 4941 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the GPCR 4941 protein.

In one embodiment, variants of a GPCR 4941 protein which function as either GPCR 4941 agonists (mimetics) or as GPCR 4941 antagonists can be identified by screening combinatorial libraries of mutants, e.g., truncation mutants, of a GPCR 4941 protein for GPCR 4941 protein agonist or antagonist activity. In one embodiment, a variegated library of GPCR 4941 variants is generated by combinatorial mutagenesis at the nucleic acid level and is encoded by a variegated gene library. A variegated library of GPCR 4941 variants can be produced by, for example, enzymatically ligating a mixture of synthetic oligonucleotides into gene sequences such that a degenerate set of potential GPCR 4941 sequences is expressible as individual polypeptides, or alternatively, as a set of larger fusion proteins (e.g., for phage display) containing the set of GPCR 4941 sequences therein. There are a variety of methods which can be used to produce libraries of potential GPCR 4941 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GPCR 4941 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a GPCR 4941 protein coding sequence can be used to generate a variegated population of GPCR 4941 fragments for screening and subsequent selection of variants of a GPCR 4941 protein. In one embodiment, a library of coding sequence fragments can be generated by treating a double stranded PCR fragment of a GPCR 4941 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the GPCR 4941 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GPCR 4941 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify GPCR 4941 variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated GPCR 4941 library. For example, a library of expression vectors can be transfected into a cell line, e.g., an endothelial or ovarian cell line, which ordinarily responds to a GPCR 4941 ligand in a particular GPCR 4941-dependent manner. The transfected cells are then contacted with a GPCR 4941 ligand and the effect of expression of the mutant on signaling by the GPCR 4941 receptor can be detected, e.g., by monitoring the generation of an intracellular second messenger (e.g., calcium, cAMP, IP3, or diacylglycerol), the phosphorylation profile of intracellular proteins, cell proliferation, differentiation and/or migration, endothelial cell angiogenic activity, tumor cell metastatic or invasive potential, the expression profile of cell surface adhesion molecules or receptors, or the activity of a GPCR 4941-regulated transcription factor. Plasmid DNA can then be recovered from the cells which score for inhibition, or alternatively, potentiation of signaling by the GPCR 4941 receptor, and the individual clones further characterized.

An isolated GPCR 4941 protein, or a portion or fragment thereof, can be used as an immunogen to generate antibodies that bind GPCR 4941 using standard techniques for polyclonal and monoclonal antibody preparation. A full-length GPCR 4941 protein can be used or, alternatively, the invention provides antigenic peptide fragments of GPCR 4941 for use as immunogens. The antigenic peptide of GPCR 4941 comprises at least 8 amino acid residues of the amino acid sequence shown in SEQ ID NO:2 and encompasses an epitope of GPCR 4941 such that an antibody raised against the peptide forms a specific immune complex with GPCR 4941. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of GPCR 4941 that are located on the surface of the protein, e.g., hydrophilic regions, as well as regions with high antigenicity.

A GPCR 4941 immunogen typically is used to prepare antibodies by immunizing a suitable subject, (e.g., rabbit, goat, mouse or other mammal) with the immunogen. An appropriate immunogenic preparation can contain, for example, recombinantly expressed GPCR 4941 protein or a chemically synthesized GPCR 4941 polypeptide. The preparation can further include an adjuvant, such as Freund's complete or incomplete adjuvant, or similar immunostimulatory agent. Immunization of a suitable subject with an immunogenic GPCR 4941 preparation induces a polyclonal anti-GPCR 4941 antibody response.

Accordingly, another aspect of the invention pertains to the use of anti-GPCR 4941 antibodies. The term “antibody” as used herein refers to immunoglobulin molecules and immunologically active portions of immunoglobulin molecules, i.e., molecules that contain an antigen binding site which specifically binds (immunoreacts with) an antigen, such as GPCR 4941. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind GPCR 4941. The term “monoclonal antibody” or “monoclonal antibody composition”, as used herein, refers to a population of antibody molecules that contain only one species of an antigen binding site capable of immunoreacting with a particular epitope of GPCR 4941. A monoclonal antibody composition thus typically displays a single binding affinity for a particular GPCR 4941 protein with which it immunoreacts.

Polyclonal anti-GPCR 4941 antibodies can be prepared as described above by immunizing a suitable subject with a GPCR 4941 immunogen. The anti-GPCR 4941 antibody titer in the immunized subject can be monitored over time by standard techniques, such as with an enzyme linked immunosorbent assay (ELISA) using immobilized GPCR 4941. If desired, the antibody molecules directed against GPCR 4941 can be isolated from the mammal (e.g., from the blood) and further purified by well known techniques, such as protein A chromatography to obtain the IgG fraction. At an appropriate time after immunization, e.g., when the anti-GPCR 4941 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem 0.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a GPCR 4941 immunogen as described above, and the culture supernatants of the resulting hybridoma cells are screened to identify a hybridoma producing a monoclonal antibody that binds GPCR 4941.

Any of the many well known protocols used for fusing lymphocytes and immortalized cell lines can be applied for the purpose of generating an anti-GPCR 4941 monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind GPCR 4941, e.g., using a standard ELISA assay.

Alternative to preparing monoclonal antibody-secreting hybridomas, a monoclonal anti-GPCR 4941 antibody can be identified and isolated by screening a recombinant combinatorial immunoglobulin library (e.g., an antibody phage display library) with GPCR 4941 to thereby isolate immunoglobulin library members that bind GPCR 4941. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-GPCR 4941 antibodies, such as chimeric and humanized monoclonal antibodies, comprising both human and non-human portions, which can be made using standard recombinant DNA techniques, can also be used in the methods of the present invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-GPCR 4941 antibody (e.g., monoclonal antibody) can be used to isolate GPCR 4941 by standard techniques, such as affinity chromatography or immunoprecipitation. An anti-GPCR 4941 antibody can facilitate the purification of natural GPCR 4941 from cells and of recombinantly produced GPCR 4941 expressed in host cells. Moreover, an anti-GPCR 4941 antibody can be used to detect GPCR 4941 protein (e.g., in a cellular lysate or cell supernatant) in order to evaluate the abundance and pattern of expression of the GPCR 4941 protein. Anti-GPCR 4941 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Regulation of GPCR 4941 Expression in Human Endothelial Cells

Human umbilical vein endothelial cells (HUVEC's) were cultured in vitro under standard conditions, described in, for example, U.S. Pat. No. 5,882,925. Experimental cultures were then exposed to either laminar shear stress (LSS) conditions or interleukin (IL)-1.

Cultured HUVEC monolayers were exposed to laminar sheer stress by culturing the cells in a specialized apparatus containing liquid culture medium. Static cultures grown in the same medium served as controls. The in vitro LSS treatment at 10 dyns/cm² was designed to simulate the shear stress generated by blood flow in a straight, healthy artery such as the internal mammary artery.

Alternatively, HUVEC cultures were treated with human IL-1β, a factor known to be involved in the inflammatory response, in order to mimic the physiologic conditions involved in the atherosclerotic state. Stimulation of endothelial cells with IL-1 induces the expression of several inflammatory markers.

Experimental and control cells were harvested and analyzed for gene expression at 1, 6 and 24 hours.

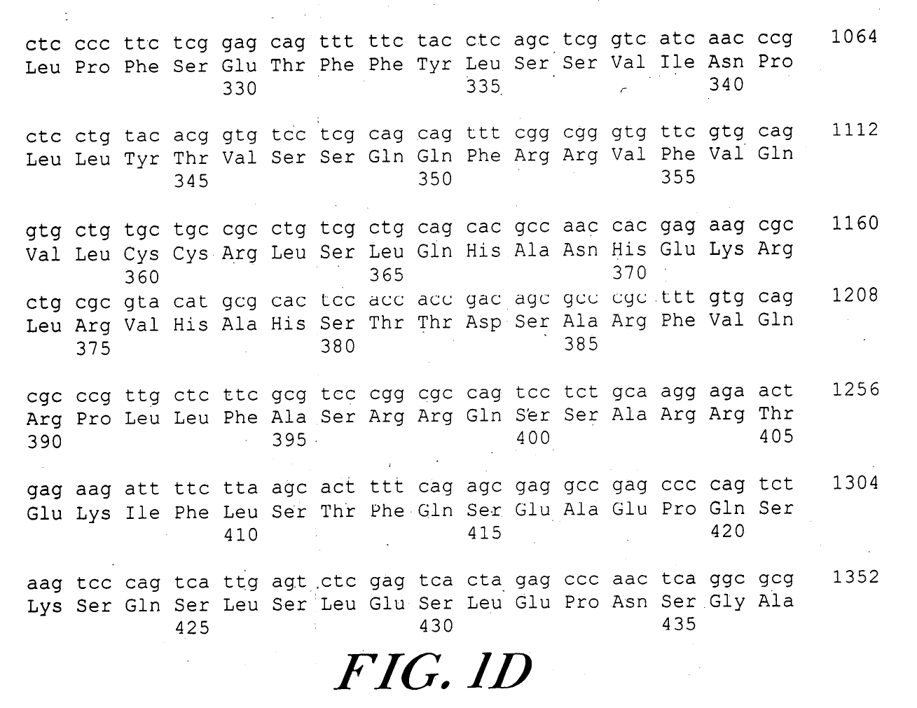

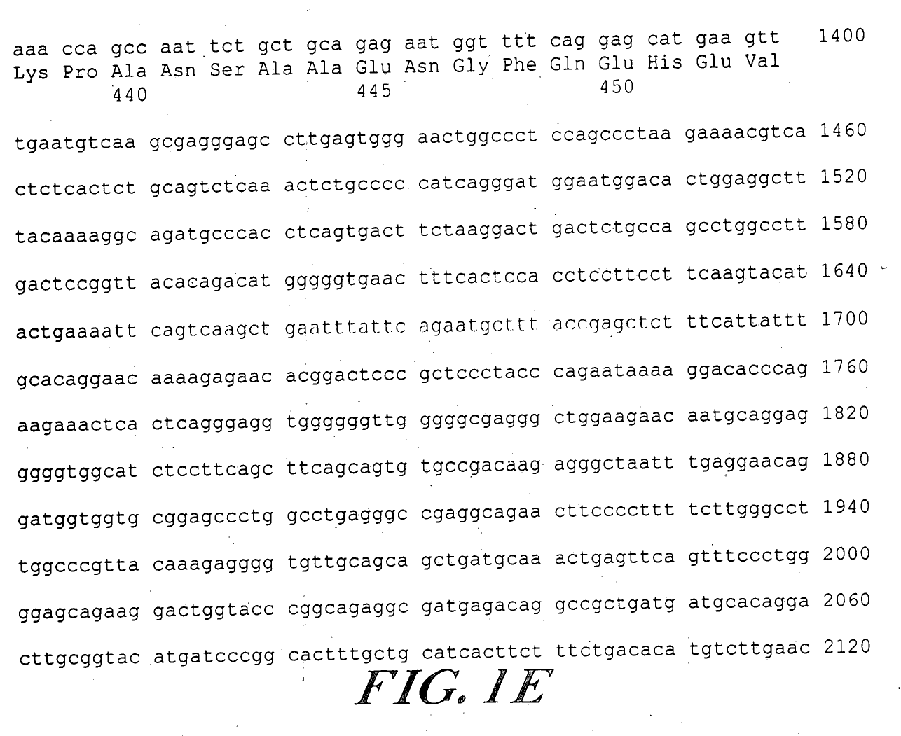

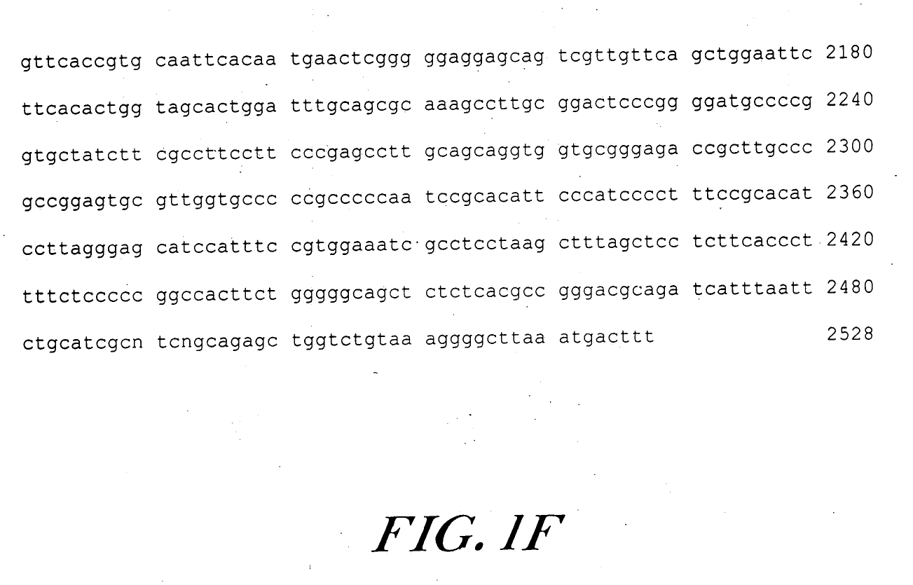

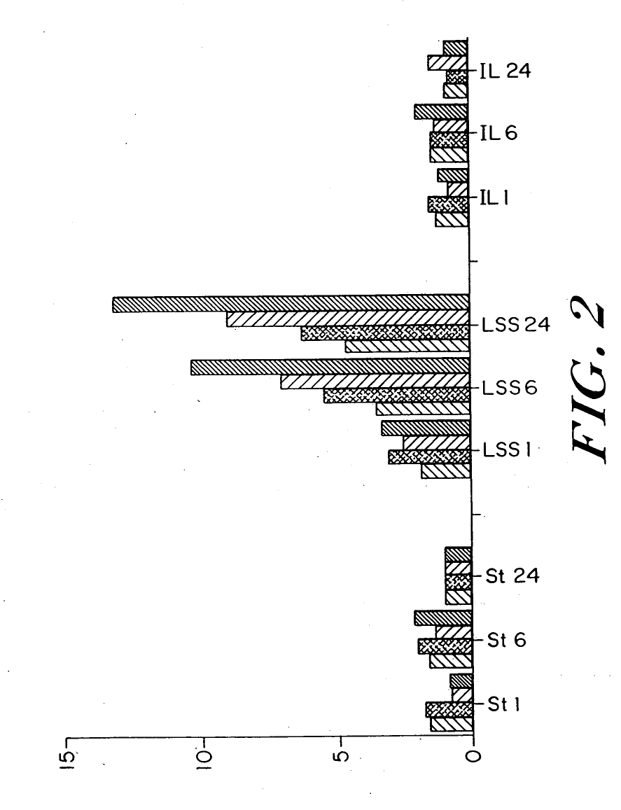

Transcriptional profiling indicated that LSS treatment of HUVECs resulted in the induction of GPCR 4941 gene expression in a time dependent fashion, with induction reaching at least 6-fold following 24 hours of LSS treatment (FIG. 2).

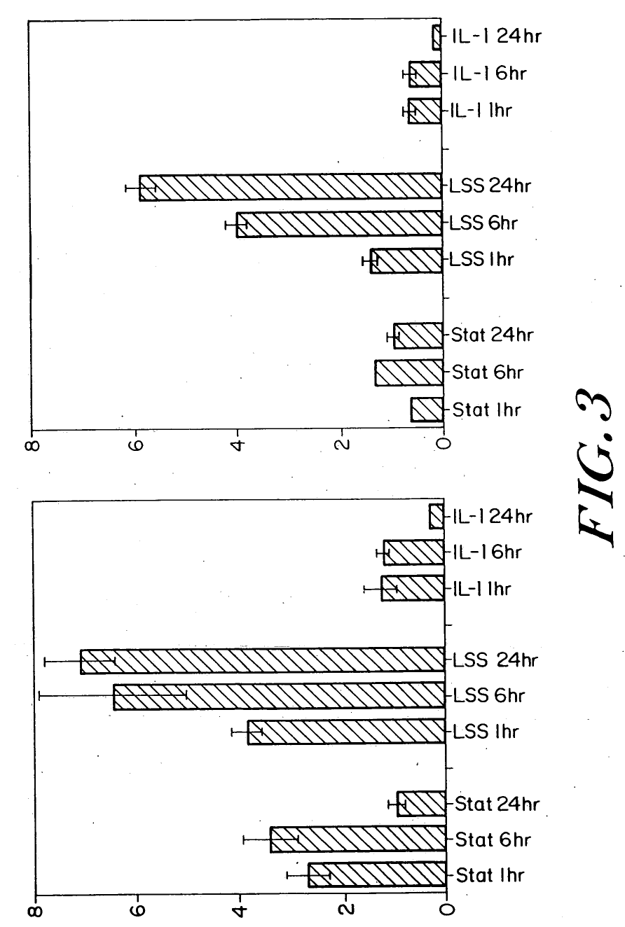

The effect of LSS and IL-1 on GPCR 4941 expression in endothelial cells was also assessed by PCR analysis. GPCR 4941 gene expression was significantly induced in HUVECs exposed to LSS. In addition, GPCR 4941 expression was reduced in HUVECs after 24 hours of IL-1 treatment (FIG. 3).

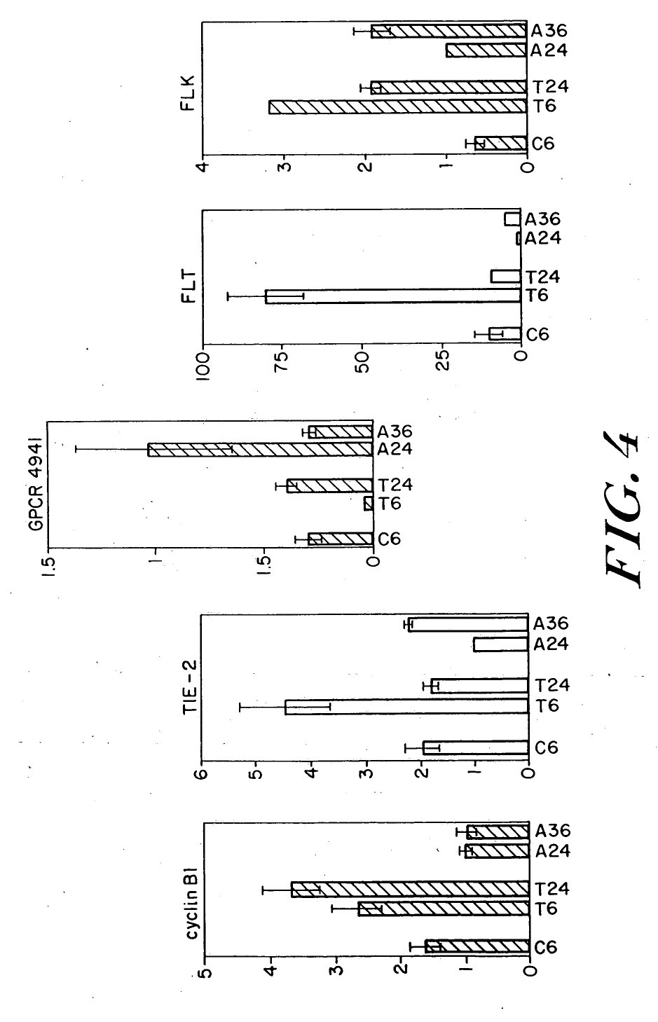

In another study, primary cultures of human microvascular endothelial cells (HMVEC) were plated on Matrigel (T) to induce tube formation, or on plastic tissue culture dishes as a control (C), and incubated for 6 or 24 hours. Samples A24 and A36 represent confluent HMVEC grown on tissue culture dishes and incubated in the absence of growth factors for 24 or 36 hours. The expression levels of GPCR 4941, as well as genes that have established roles in cell growth and angiogenesis were assessed by PCR. Genes such as cyclin B 1, TIE-2, FLT and FLK were up-regulated after 6 hours of incubation on Matrigel (T6), at which time the cells are performing angiogenesis-related functions, e.g., proliferation, migration and tube formation (FIG. 4). In contrast, the expression of GPCR 4941 was significantly down-regulated in the T6 sample.

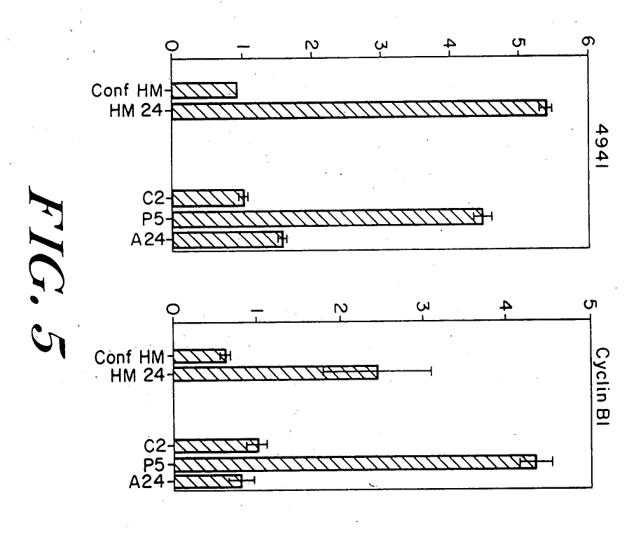

Furthermore, the expression of GPCR 4941 was also determined in proliferating HUVEC and HMVEC cells growth on plastic tissue culture dishes. Similar to cyclin B 1, GPCR 4941 was upregulated in rapidly proliferating endothelial cells (HMVEC and HUVEC) relative to confluent and growth factor depleted endothelial cells that are in a quiescent state (FIG. 5).

Collectively, these data indicate that GPCR 4941 may be involved in the regulation of endothelial cell processes such as growth, proliferation, differentiation, migration and tube formation.

### Example 2

### Regulation of GPCR 4941 Expression in Apo E Knockout Animals

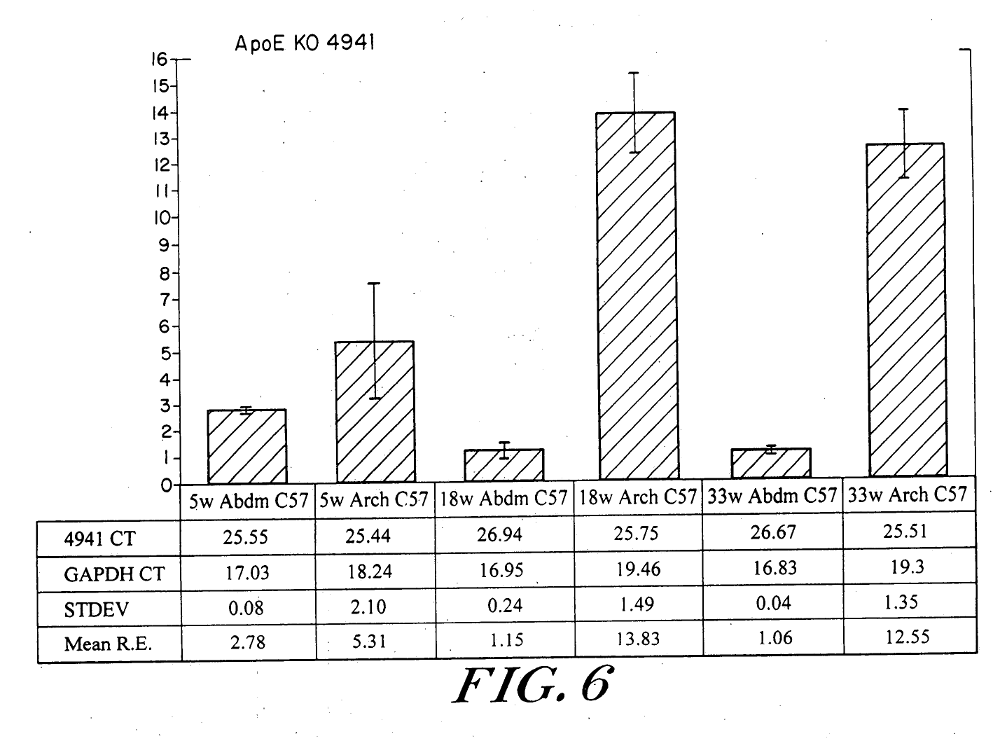

The expression of GPCR 4941 was assessed in C57 ApoE knockout animals at 5, 18 and 33 weeks of age. In the ApoE knockout animals, the aortic arch region is prone to formation of atherosclerotic lesions, whereas the abdominal aorta is typically free of such lesions. At 5 weeks of age lesion development is minimal, whereas by 18 weeks of age complex lesion formation is observed, which persists at 33 weeks of age. GPCR 4941 expression was analyzed in tissue sections dissected from either the abdominal aorta or the aortic arch region. GPCR 4941 is upregulated in the aortic arch region as compared to the abdominal aorta at all ages tested (FIG. 6), indicating a correlation with the pathogenesis of atherosclerosis.

### Example 3

### Regulation of GPCR 4941 Expression in Human Tumors

The expression of GPCR 4941 in-human tumors was analyzed by TaqMan® Quantitative Polymerase Chain Reaction.

Probes were designed by PrimerExpress software (PE Biosystems) based on the sequence of the GPCR 4941 gene. Each GPCR 4941 gene probe was labeled using FAM (6-carboxyfluorescein), and the β2-microglobulin reference probe was labeled with a different fluorescent dye, VIC. The differential labeling of the target gene and internal reference gene, thus, enabled measurement in the same well. Forward and reverse primers and probes for both the P2-microglobulin and the target gene were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM of probe for β-2 microglobulin and 600 nM of forward and reverse primers plus 200 nM of probe for the target gene. TaqMan matrix experiments were carried out using an ABI PRISM 7700 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds followed by 60° C. for 1 minute.

A comparative Ct method was used for the relative quantitation of gene expression. The following method was used to quantitatively calculate GPCR 4941 gene expression in the various samples relative to β-2 microglobulin expression in the same sample. The threshold cycle (Ct) value was defined as the cycle at which a statistically significant increase in fluorescence is detected. A lower Ct value was indicative of a higher mRNA concentration. The Ct value of the GPCR 4941 gene was normalized by subtracting the Ct value of the β-2 microglobulin gene to obtain a ΔCt value using the following formula:

ΔCt=Ct_{GPCR4941}−Ct_{β-2 microglobulin}

Expression was then calibrated against a cDNA control sample containing no template. The ΔCt value for the calibrator sample was then subtracted from ΔCt for each tissue sample according to the following formula:

ΔΔCt=ΔCt-ₛₐₘₚₑ−ΔCt-_{calibrator}

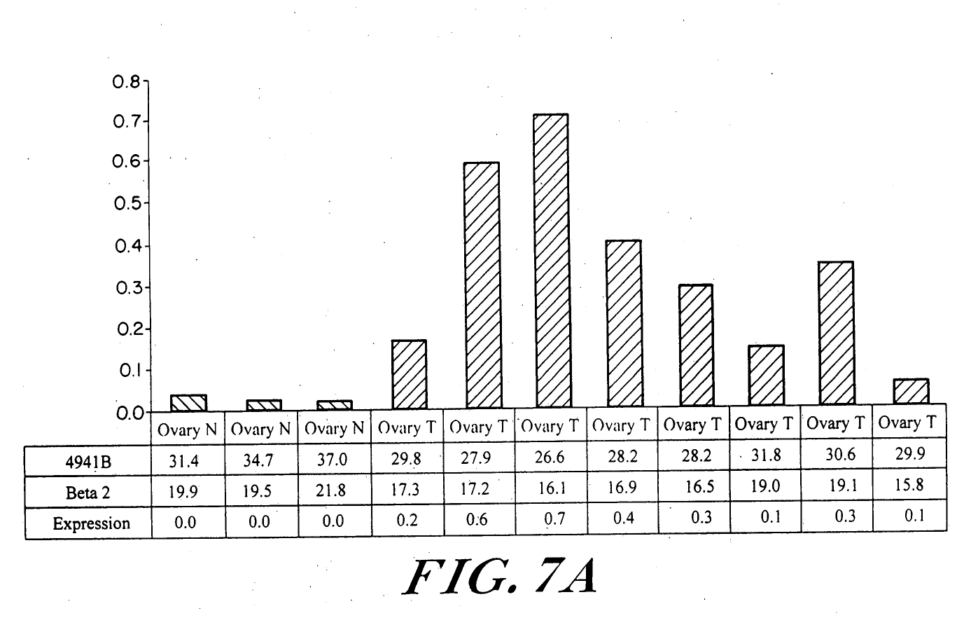

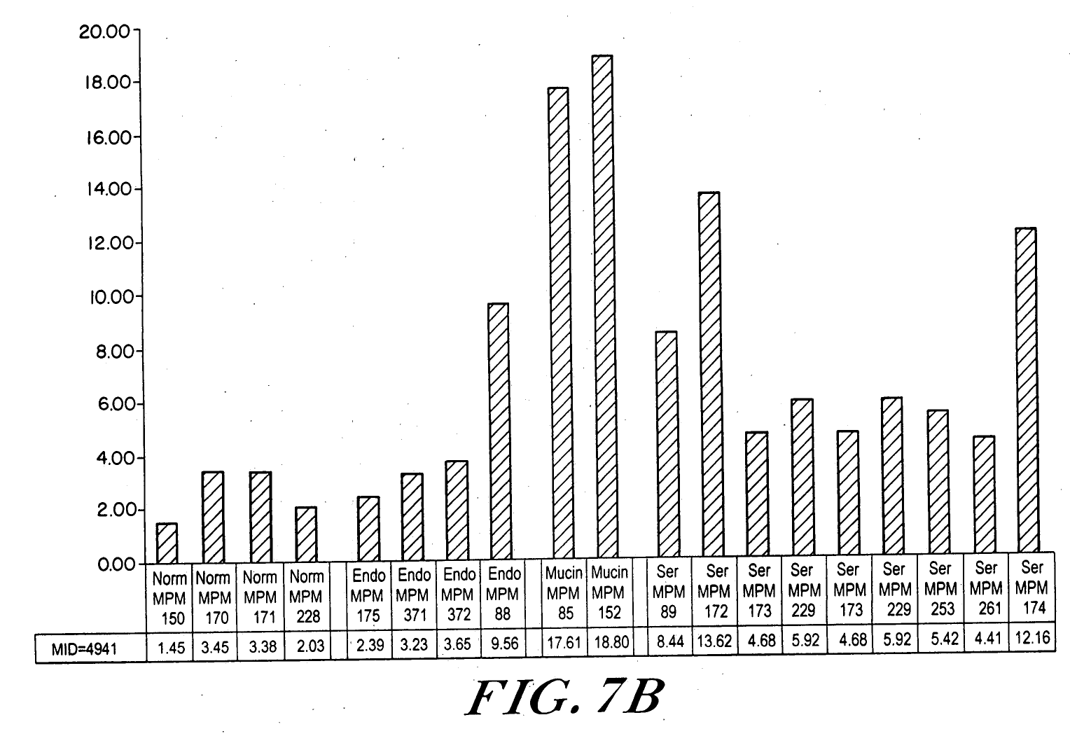

Relative expression was then calculated using the arithmetic formula given by 2^{-ΔΔCt-}. As shown in FIG. 7A, GPCR 4941 gene expression was up-regulated in 7 out of 8 ovarian serous tumor samples as compared to control normal ovary samples. Moreover, transcriptional profiling experiments revealed that GPCR 4941 expression was increased in 1 out of 4 endometrioid, 2 out of 2 mucinous, and 9 out of 9 serous type ovarian tumors as compared to normal ovarian epithelial samples (FIG. 7B). Furthermore, in situ hybridization experiments revealed that GPCR 4941 was strongly expressed in ovarian papillary serous tumors and was localized to the epithelial cells of the tumors. Taken together, these data indicate that GPCR 4941 may play a role in the growth regulation and progression of ovarian carcinomas.

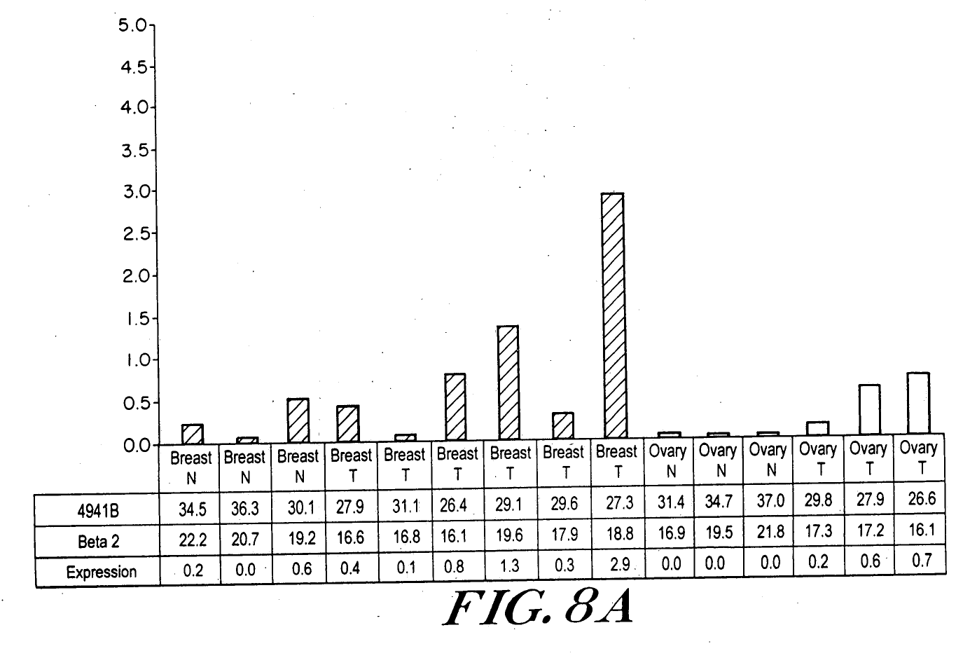

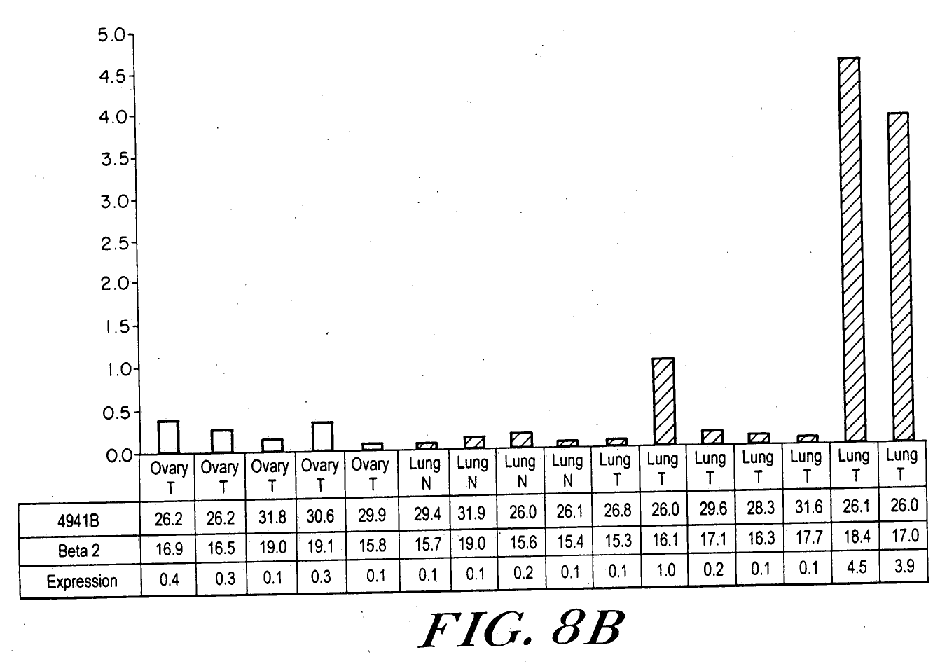

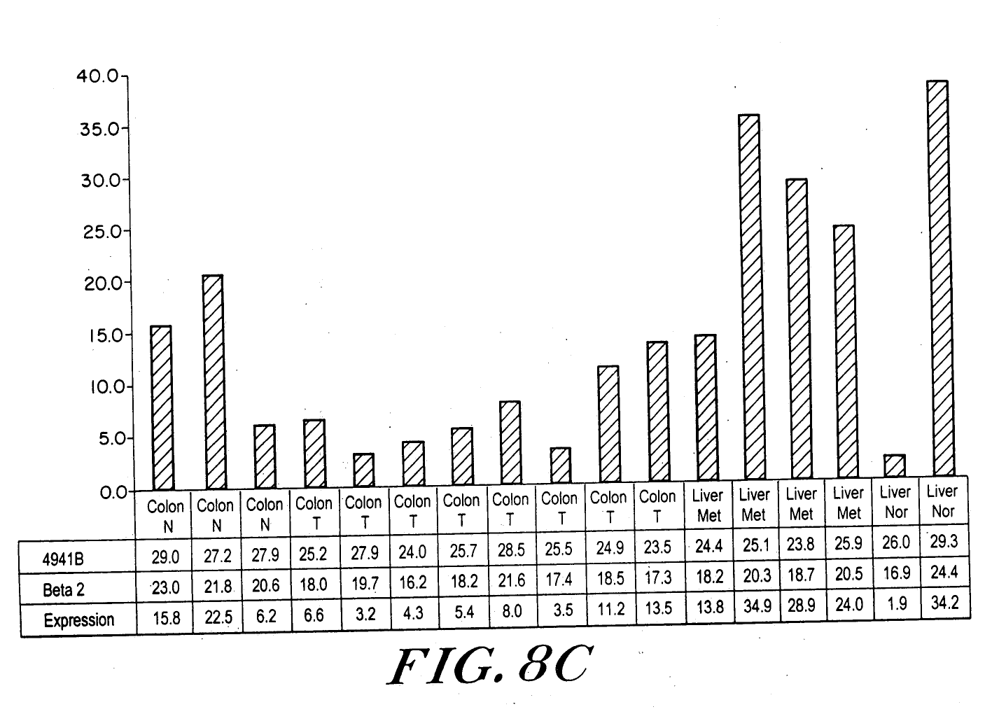

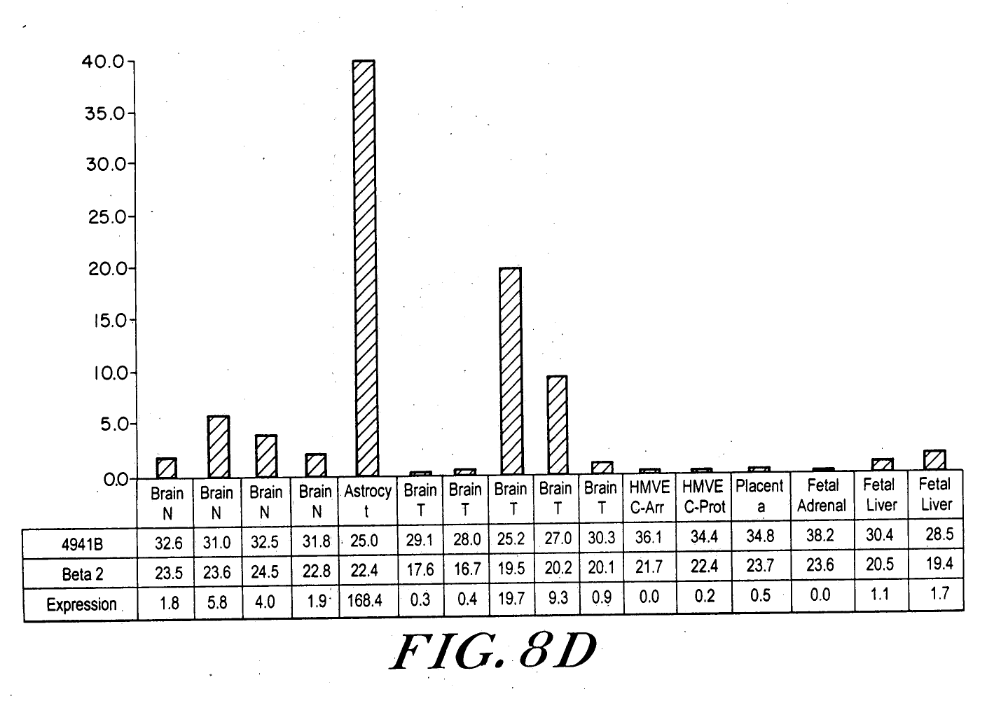

GPCR 4941 gene expression was also upregulated in 3 out of 6 breast tumor samples (intraductal carcinoma and ductal carcinoma in situ), 3 out of 7 lung tumor samples (squamous cell carcinoma), and 2 out of 5 brain tumors (glioblastoma), as compared to clinical normal tissue samples (see FIGS. 8A and 8B).

In situ hybridization analysis revealed that within glioblastomas, GPCR 4941 is expressed in malignant gial and tumor endothelial cells.

Thus, modulation of GPCR 4941 receptor activation and/or GPCR 4941 mediated signal transduction may be of therapeutic importance in the inhibition of tumorigenesis.

### Example 4

### GPCR 4941 Expression in Human Tissues

The expression of GPCR 4941 in human tissues and cell lines was analyzed by TaqMan® Quantitative Polymerase Chain Reaction, as described above.

GPCR4941 was strongly expressed in the brain, colon, kidney, fetal heart and fetal liver, as well as in cultured astrocytes and epithelial cells. GPCR 4941 was also expressed in prostate epithelial cells, spinal cord, brain cortex, hypothalamus, glioblastoma, breast, prostate, liver, lung, tonsil, lymph node, skeletal muscle, dermal fibroblasts, skin, osteoblasts (primary, undifferentiated and differentiated), and osteoclasts.

The expression of GPCR 4941 was modulated in pathological states affecting the colon (e.g., upregulated in colon tumor and downregulated in ischemic bowel disease [IBD]) and lung (e.g., upregulated in lung tumor and chronic obstructive pulmonary disease [COPD]). Moreover, GPCR 4941 expression was downregulated in late passage aortic smooth muscle cells as compared to early passage cells.

Northern blot analysis with human tissue RNA samples revealed that GPCR 4941 mRNA was expressed in the brain, prostate, ovary, heart, lung, and neurons. Within the brain, in situ hybridization analysis revealed GPCR 4941 expression in neurons and astrocytes.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a nucleic acid molecule associated with a cardiovascular or tumorigenic disorder comprising:
a) contacting a sample comprising nucleic acid molecules with a hybridization probe comprising at least 25 contiguous nucleotides of SEQ ID NO:1; and
b) detecting the presence of a nucleic acid molecule in said sample that hybridizes to said probe, thereby identifying a nucleic acid molecule associated with a cardiovascular or tumorigenic disorder.

2. The method of claim 1, wherein said hybridization probe is detectably labeled.

3. The method of claim 1, wherein said sample comprising nucleic acid molecules is subjected to agarose gel electrophoresis and southern blotting prior to contacting with said hybridization probe.

4. The method of claim 1, wherein said sample comprising nucleic acid molecules is subjected to agarose gel electrophoresis and northern blotting prior to contacting with said hybridization probe.

5. The method of claim 1, wherein said detecting is by in situ hybridization.

6. A method of identifying a nucleic acid associated with a cardiovascular or tumorigenic disorder comprising:
a) contacting a sample comprising nucleic acid molecules with a first and a second amplification primer, said first primer comprising at least 25 contiguous nucleotides of SEQ ID NO:1 and said second primer comprising at least 25 contiguous nucleotides from the complement of SEQ ID NO:1;
b) incubating said sample under conditions that allow nucleic acid amplification; and
c) detecting the presence of a nucleic acid molecule in said sample that is amplified, thereby identifying a nucleic acid molecule associated with a cardiovascular or tumorigenic disorder.

7. The method of claim 6, wherein said sample comprising nucleic acid molecules is subjected to agarose gel electrophoresis after said incubation step.

8. The method of any one of claims 1 or 6, wherein said method is used to detect mRNA in said sample.

9. The method of any one of claims 1 or 6, wherein said method is used to detect genomic DNA in said sample.

10. A method of identifying a polypeptide associated with a cardiovascular or tumorigenic disorder comprising:
a) contacting a sample comprising polypeptides with a GPCR 4941 binding substance; and
b) detecting the presence of a polypeptide in said sample that binds to said GPCR 4941 binding substance, thereby identifying a polypeptide associated with a cardiovascular or tumorigenic disorder.

11. The method of claim 10, wherein said binding substance is an antibody.

12. The method of claim 10, wherein said binding substance is detectably labeled.

13. A method of identifying a subject having a cardiovascular or tumorigenic disorder, or at risk for developing a cardiovascular or tumorigenic disorder comprising:
a) contacting a sample obtained from said subject comprising nucleic acid molecules with a hybridization probe comprising at least 25 contiguous nucleotides of SEQ ID NO:1; and
b) detecting the presence of a nucleic acid molecule in said sample that hybridizes to said probe, thereby identifying a subject having a cardiovascular or tumorigenic disorder, or at risk for developing a cardiovascular or tumorigenic disorder.

14. The method of claim 13, wherein said hybridization probe is detectably labeled.

15. The method of claim 13, wherein said sample comprising nucleic acid molecules is subjected to agarose gel electrophoresis and southern blotting prior to contacting with said hybridization probe.

16. The method of claim 13, wherein said sample comprising nucleic acid molecules is subjected to agarose gel electrophoresis and northern blotting prior to contacting with said hybridization probe.

17. The method of claim 13, wherein said detecting is by in situ hybridization.

18. A method of identifying a subject having a cardiovascular or tumorigenic disorder or at risk for developing a cardiovascular or tumorigenic disorder comprising:
a) contacting a sample obtained from said subject comprising nucleic acid molecules with a first and a second amplification primer, said first primer comprising at least 25 contiguous nucleotides of SEQ ID NO:1 and said second primer comprising at least 25 contiguous nucleotides from the complement of SEQ ID NO:1;
b) incubating said sample under conditions that allow nucleic acid amplification; and
c) detecting the presence of a nucleic acid molecule in said sample that is amplified, thereby identifying a subject having a cardiovascular or tumorigenic disorder, or at risk for developing a cardiovascular or tumorigenic disorder.

19. The method of claim 18, wherein said sample comprising nucleic acid molecules is subjected to agarose gel electrophoresis after said incubation step.

20. The method of any one of claims 13 or 18, wherein said method is used to detect mRNA in said sample.

21. The method of any one of claims 13 or 18, wherein said method is used to detect genomic DNA in said sample.

22. A method of identifying a subject having a cardiovascular or tumorigenic disorder, or at risk for developing a cardiovascular or tumorigenic disorder comprising:
a) contacting a sample obtained from said subject comprising polypeptides with a GPCR 4941 binding substance; and
b) detecting the presence of a polypeptide in said sample that binds to said GPCR 4941 binding substance, thereby identifying a subject having a cardiovascular or tumorigenic disorder, or at risk for developing a cardiovascular or tumorigenic disorder.

23. The method of claim 22, wherein said binding substance is an antibody.

24. The method of claim 22, wherein said binding substance is detectably labeled.

25. A method for identifying a compound capable of treating a cardiovascular or tumorigenic disorder characterized by aberrant GPCR 4941 nucleic acid expression or GPCR 4941 polypeptide activity comprising assaying the ability of the compound to modulate GPCR 4941 nucleic acid expression or GPCR 4941 polypeptide activity, thereby identifying a compound capable of treating a cardiovascular or tumorigenic disorder characterized by aberrant GPCR 4941 nucleic acid expression or GPCR 4941 polypeptide activity.

26. The method of claim 25, wherein the disorder a disorder associated with aberrant angiogenesis.

27. The method of claim 25, wherein the disorder is a disorder associated with aberrant vascularization.

28. The method of claim 25, wherein the disorder is atherosclerosis.

29. The method of claim 25, wherein the disorder is ovarian cancer.

30. The method of claim 25, wherein the ability of the compound to modulate the activity of the GPCR 4941 polypeptide is determined by detecting the induction of an intracellular second messenger.

31. A method for treating a subject having a cardiovascular or tumorigenic disorder characterized by aberrant GPCR 4941 polypeptide activity or aberrant GPCR 4941 nucleic acid expression comprising administering to the subject a GPCR 4941 modulator, thereby treating said subject having a cardiovascular or tumorigenic disorder.

32. The method of claim 31, wherein the GPCR 4941 modulator is a small molecule.

33. The method of claim 31, wherein the disorder is a disorder associated with aberrant angiogenesis.

34. The method of claim 31, wherein the disorder is a disorder associated with aberrant vascularization.

35. The method of claim 31, wherein the disorder is atherosclerosis.

36. The method of claim 31, wherein the disorder is ovarian cancer.

37. The method of claim 31, wherein said GPCR 4941 modulator is administered in a pharmaceutically acceptable formulation.

38. The method of claim 31, wherein said GPCR 4941 modulator is administered using a gene therapy vector.

39. The method of 31, wherein the GPCR 4941 modulator is capable of modulating GPCR 4941 polypeptide activity.

40. The method of claim 39, wherein the GPCR 4941 modulator is an anti-GPCR 4941 antibody.

41. The method of claim 39, wherein the GPCR 4941 modulator is a GPCR 4941 polypeptide comprising the amino acid sequence of SEQ ID NO:2, or a fragment thereof.

42. The method of claim 39, wherein the GPCR 4941 modulator is a GPCR 4941 polypeptide comprising an amino acid sequence which is at least 90 percent identical to the amino acid sequence of SEQ ID NO:2, wherein said percent identity is calculated using the ALIGN program for comparing amino acid sequences, a PAM120 weight residue table, a gap length penalty of 12, and a gap penalty of 4.

43. The method of claim 39, wherein the GPCR 4941 modulator is an isolated naturally occurring allelic variant of a polypeptide consisting of the amino acid sequence of SEQ ID NO:2, wherein the polypeptide is encoded by a nucleic acid molecule which hybridizes to a complement of a nucleic acid molecule consisting of SEQ ID NO:1 at 4×SSC at 65-70° C. followed by one or more washes in 1×SSC, at 65-70° C.

44. The method of claim 31, wherein the GPCR 4941 modulator is capable of modulating GPCR 4941 nucleic acid expression.

45. The method of claim 44, wherein the GPCR 4941 modulator is an antisense GPCR 4941 nucleic acid molecule.

46. The method of claim 44, wherein the GPCR 4941 modulator is a ribozyme.

47. The method of claim 44, wherein the GPCR 4941 modulator comprises the nucleotide sequence of SEQ ID NO:1, or a fragment thereof.

48. The method of claim 44, wherein the GPCR 4941 modulator comprises a nucleic acid molecule encoding a polypeptide comprising an amino acid sequence which is at least 90 percent identical to the amino acid sequence of SEQ ID NO:2, wherein said percent identity is calculated using the ALIGN program for comparing amino acid sequences, a PAM120 weight residue table, a gap length penalty of 12, and a gap penalty of 4.

49. The method of claim 44, wherein the GPCR 4941 modulator comprises a nucleic acid molecule encoding a naturally occurring allelic variant of a polypeptide comprising the amino acid sequence of SEQ ID NO:2, wherein the nucleic acid molecule which hybridizes to a complement of a nucleic acid molecule consisting of SEQ ID NO:1 at 0.4×SSC at 65-70° C. followed by one or more washes in 1×SSC, at 65-70° C.

50. A method for identifying a: compound capable of modulating an endothelial cell activity comprising:
a) contacting an endothelial cell with a test compound; and
b) assaying the ability of the test compound to modulate the expression of a GPCR 4941 nucleic acid or the activity of a GPCR 4941 polypeptide; thereby identifying a compound capable of modulating an endothelial cell activity.

51. The method of claim 50, wherein said endothelial cell activity is cell proliferation.

52. The method of claim 50, wherein said endothelial cell activity is cell migration.

53. The method of claim 50, wherein said endothelial cell activity is expression of cell surface adhesion molecules.

54. A method for modulating an endothelial cell activity comprising contacting an endothelial cell with a GPCR 4941 modulator, thereby modulating said endothelial cell activity.

55. The method of claim 54, wherein the GPCR 4941 modulator is a small molecule.

56. The method of claim 54, wherein said endothelial cell activity is cell proliferation.

57. The method of claim 54, wherein said endothelial cell activity is cell migration.

58. The method of claim 54, wherein said endothelial cell activity is expression of cell surface adhesion molecules.

59. The method of claim 54, wherein the GPCR 4941 modulator is capable of modulating GPCR 4941 polypeptide activity.

60. The method of claim 59, wherein the GPCR 4941 modulator is an anti-GPCR 4941 antibody.

61. The method of claim 59, wherein the GPCR 4941 modulator is a GPCR 4941 polypeptide comprising the amino acid sequence of SEQ ID NO:2, or a fragment thereof.

62. The method of claim 59, wherein the GPCR 4941 modulator is a GPCR 4941 polypeptide comprising an amino acid sequence which is at least 90 percent identical to the amino acid sequence of SEQ ID NO:2, wherein said percent identity is calculated using the ALIGN program for comparing amino acid sequences, a PAM120 weight residue table, a gap length penalty of 12, and a gap penalty of 4.

63. The method of claim 59, wherein the GPCR 4941 modulator is an isolated naturally occurring allelic variant of a polypeptide consisting of the amino acid sequence of SEQ ID NO:2, wherein the polypeptide is encoded by a nucleic acid molecule which hybridizes to a complement of a nucleic acid molecule consisting of SEQ ID NO:1 at 4×SSC at 65-70° C. followed by one or more washes in 1×SSC, at 65-70° C.

64. The method of claim 54, wherein the GPCR 4941 modulator is capable of modulating GPCR 4941 nucleic acid expression.

65. The method of claim 64, wherein the GPCR 4941 modulator is an antisense GPCR 4941 nucleic acid molecule.

66. The method of claim 64, wherein the GPCR 4941 modulator is a ribozyme.

67. The method of claim 64, wherein the GPCR 4941 modulator comprises the nucleotide sequence of SEQ ID NO:1, or a fragment thereof.

68. The method of claim 64, wherein the GPCR 4941 modulator comprises a nucleic acid molecule encoding a polypeptide comprising an amino acid sequence which is at least 90 percent identical to the amino acid sequence of SEQ ID NO:2, wherein said percent identity is calculated using the ALIGN program for comparing amino acid sequences, a PAM120 weight residue table, a gap length penalty of 12, and a gap penalty of 4.

69. The method of claim 64, wherein the GPCR 4941 modulator comprises a nucleic acid molecule encoding a naturally occurring allelic variant of a polypeptide comprising the amino acid sequence of SEQ ID NO:2, wherein the nucleic acid molecule which hybridizes to a complement of a nucleic acid molecule consisting of SEQ ID NO:1 at 4×SSC at 65-70° C. followed by one or more washes in 1×SSC, at 65-70° C.


SEQUENCE LISTING (3 sequences)

SEQ ID NO: 1 - DNA, 1362 bp, Homo sapiens
  CDS (1)..(1359)
atggcttcacccagcctcccgggcagtgactgctcccaaatcattgatcacagtcatgtccccgagtttgaggtggccacctggatcaaaatcacccttattctggtgtacctgatcatcttcgtgatgggccttctggggaacagcgccaccattcgggtcacccaggtgctgcagaagaaaggatacttgcagaaggaggtgacagaccacatggtgagtttggcttgctcggacatcttggtgttcctcatcggcatgcccatggagttctacagcatcatctggaatcccctgaccacgtccagctacaccctgtcctgcaagctgcacactttcctcttcgaggcctgcagctacgctacgctgctgcacgtgctgacactcagctttgagcgctacatcgccatctgtcaccccttcaggtacaaggctgtgtcgggaccttgccaggtgaagctgctgattggcttcgtctgggtcacctccgccctggtggcactgcccttgctgtttgccatgggtactgagtaccccctggtgaacgtgcccagccaccggggtctcacttgcaaccgctccagcacccgccaccacgagcagcccgagacctccaatatgtccatctgtaccaacctctccagccgctggaccgtgttccagtccagcatcttcggcgccttcgtggtctacctcgtggtcctgctctccgtagccttcatgtgctggaacatgatgcaggtgctcatgaaaagccagaagggctcgctggccgggggcacgcggcctccgcagctgaggaagtccgagagcgaagagagcaggaccgccaggaggcagaccatcatcttcctgaggctgattgttgtgacattggccgtatgctggatgcccaaccagattcggaggatcatggctgcggccaaacccaagcacgactggacgaggtcctacttccgggcgtacatgatcctcctccccttctcggagacgtttttctacctcagctcggtcatcaacccgctcctgtacacggtgtcctcgcagcagtttcggcgggtgttcgtgcaggtgctgtgctgccgcctgtcgctgcagcacgccaaccacgagaagcgcctgcgcgtacatgcgcactccaccaccgacagcgcccgctttgtgcagcgcccgttgctcttcgcgtcccggcgccagtcctctgcaaggagaactgagaagattttcttaagcacttttcagagcgaggccgagccccagtctaagtcccagtcattgagtctcgagtcactagagcccaactcaggcgcgaaaccagccaattctgctgcagagaatggttttcaggagcatgaagtttga

SEQ ID NO: 2 - protein, 453 aa, Homo sapiens
MASPSLPGSDCSQIIDHSHVPEFEVATWIKITLILVYLIIFVMGLLGNSATIRVTQVLQKKGYLQKEVTDHMVSLACSDILVFLIGMPMEFYSIIWNPLTTSSYTLSCKLHTFLFEACSYATLLHVLTLSFERYIAICHPFRYKAVSGPCQVKLLIGFVWVTSALVALPLLFAMGTEYPLVNVPSHRGLTCNRSSTRHHEQPETSNMSICTNLSSRWTVFQSSIFGAFVVYLVVLLSVAFMCWNMMQVLMKSQKGSLAGGTRPPQLRKSESEESRTARRQTIIFLRLIVVTLAVCWMPNQIRRIMAAAKPKHDWTRSYFRAYMILLPFSETFFYLSSVINPLLYTVSSQQFRRVFVQVLCCRLSLQHANHEKRLRVHAHSTTDSARFVQRPLLFASRRQSSARRTEKIFLSTFQSEAEPQSKSQSLSLESLEPNSGAKPANSAAENGFQEHEV

SEQ ID NO: 3 - DNA, 2528 bp, Homo sapiens
  CDS (42)..(1400)
  N at positions 2490 and 2493 may be Adenine, Guanine, Cytosine or Thymine
gggagtcgacccacgcgtccggtagcctggtgctctttctcatggcttcacccagcctcccgggcagtgactgctcccaaatcattgatcacagtcatgtccccgagtttgaggtggccacctggatcaaaatcacccttattctggtgtacctgatcatcttcgtgatgggccttctggggaacagcgccaccattcgggtcacccaggtgctgcagaagaaaggatacttgcagaaggaggtgacagaccacatggtgagtttggcttgctcggacatcttggtgttcctcatcggcatgcccatggagttctacagcatcatctggaatcccctgaccacgtccagctacaccctgtcctgcaagctgcacactttcctcttcgaggcctgcagctacgctacgctgctgcacgtgctgacactcagctttgagcgctacatcgccatctgtcaccccttcaggtacaaggctgtgtcgggaccttgccaggtgaagctgctgattggcttcgtctgggtcacctccgccctggtggcactgcccttgctgtttgccatgggtactgagtaccccctggtgaacgtgcccagccaccggggtctcacttgcaaccgctccagcacccgccaccacgagcagcccgagacctccaatatgtccatctgtaccaacctctccagccgctggaccgtgttccagtccagcatcttcggcgccttcgtggtctacctcgtggtcctgctctccgtagccttcatgtgctggaacatgatgcaggtgctcatgaaaagccagaagggctcgctggccgggggcacgcggcctccgcagctgaggaagtccgagagcgaagagagcaggaccgccaggaggcagaccatcatcttcctgaggctgattgttgtgacattggccgtatgctggatgcccaaccagattcggaggatcatggctgcggccaaacccaagcacgactggacgaggtcctacttccgggcgtacatgatcctcctccccttctcggagacgtttttctacctcagctcggtcatcaacccgctcctgtacacggtgtcctcgcagcagtttcggcgggtgttcgtgcaggtgctgtgctgccgcctgtcgctgcagcacgccaaccacgagaagcgcctgcgcgtacatgcgcactccaccaccgacagcgcccgctttgtgcagcgcccgttgctcttcgcgtcccggcgccagtcctctgcaaggagaactgagaagattttcttaagcacttttcagagcgaggccgagccccagtctaagtcccagtcattgagtctcgagtcactagagcccaactcaggcgcgaaaccagccaattctgctgcagagaatggttttcaggagcatgaagtttgaatgtcaagcgagggagccttgagtgggaactggccctccagccctaagaaaacgtcactctcactctgcagtctcaaactatgcccccatcagggatggaatggacactggaggctttacaaaaggcagatgcccacctcagtgacttctaaggactgactctgccagcctggccttgactccggttacacagacatgggggtgaactttcactccacctccttccttcaagtacatactgaaaattcagtcaagctgaatttattcagaatgctttaccgagctctttcattatttgcacaggaacaaaagagaacacggactcccgctccctacccagaataaaaggacacccagaagaaactcactcagggaggtggggggttgggggcgagggctggaagaacaatgcaggagggggtggcatctccttcagcttcagcagtgtgccgagaagagggctaatttgaggaacaggatggtggtgcggagccctggcctgagggccgaggcagaacttccccttttcttgggccttggcccgttacaaagaggggtgttgcagcagctgatgcaaactgagttcagtttccctggggagcagaaggactggtacccggcagaggcgatgagacaggccgctgatgatgcacaggacttgcggtacatgatcccggcactttgctccatcacttctttctgacacatgtcttgaacgttcaccgtgcaattcacaatgaactcgggggaggagcagtcgttgttcagctggaattcttcacactggtagcactggatttgcagcgcaaagccttgcggactcccggggatgccccggtgctatcttcgccttccttcccgagccttgcagcaggtggtgcgggagaccgcttgcccgccggagtgcgttggtgcccccgcccccaatccgcacattcccatcccctttccgcacatccttagggagcatccatttccgtggaaatcgcctcctaagctttagctcctcttcacccttttctcccccggccacttctgggggcagctctctcacgccgggacgcagatcatttaattctgcatcgcntcngcagagctggtctgtaaaggggcttaaatgacttt